(19) United States
(12) Patent Application Publication
(10) Pub. No.: US 2016/0195524 A1
(43) Pub. Date: Jul. 7, 2016

(54) Automated Assay

(71) Applicant: University of Strathclyde, Glasgow (GB)

(72) Inventors: David COWAN, Glasgow (GB); William STIMSON, Glasglow (GB)

(21) Appl. No.: 14/913,068

(22) PCT Filed: Jan. 27, 2015
(86) PCT No.: PCT/GB2015/050181

§ 371 Date: Feb. 19, 2016

(30) Foreign Application Priority Data
Jan. 28, 2014 (GB) 1401426.0

(51) Int. Cl. G01N 33/543; G01N 33/78; G01N 33/569

(52) U.S. Cl. CPC G01N 33/54386; G01N 33/56916; G01N 33/78; G01N 2333/575; G01N 2333/255; G01N 2469/10

(57) ABSTRACT

Disclosed is a system and method for automating the performance of a conjugate or sandwich assay, and a cassette for use with the system. The cassette comprises a fluid conduit and one or more chambers in the fluid conduit, from which a measurement may be acquired from a sample using a cassette reader device. Sample and reagent fluids may be caused to flow along the fluid conduit to perform the assay and control over the progress of the assay may be exerted at least in part by the arrangement of chambers in the fluid conduit. Also disclosed is a tablet or bead for use with the assay system, which may be incorporated in the cassette. The tablet or bead may comprise one or more reagents to be used in the assay, in a soluble matrix. Use of an acridan or acridinium ester label may enable a sensitive measurement to be rapidly acquired. The assay may be configured to be performed by a clinician at the point of use or care.

### FIELD OF THE INVENTION

The invention relates to the field of devices and methods for performing assays, such as immunoassays. In particular, the invention relates to automated quantitative assays suitable for point of care/use applications.

### BACKGROUND TO THE INVENTION

An assay is an analytical procedure for qualitatively or quantitatively assessing the presence, amount or the functional activity of a target entity (known as an analyte) in a sample, such as a biological sample. Assays are common laboratory procedures in the medical, pharmacological, environmental/molecular biological fields, used to detect analytes such as drug compounds, biochemical substances or particular cell types.

Many assays, particularly those used to make quantitative measurement of an analyte, are complex procedures, which must be performed by skilled personnel. As such, they can be time consuming and expensive to conduct, and samples to be assayed are often sent away to dedicated laboratory facilities, with results available days or weeks later.

“Point of care” diagnostic tests (or, for non-clinical applications, “point of use” testing) can convey certain benefits over laboratory tests.

The terms “point of care” and “point of use” are generally understood to include diagnostic tests/assays in which samples, such as patient specimens, are assayed at or near the sampling location such that completion of the assay and any follow-up action based on the results can be completed within the same patient/problem encounter.

Medical/veterinary applications of point of care testing include intensive/emergency care encounters, tests conducted in hospital wards, general practice, outpatient clinics, veterinary surgeries and the like. Non-medical applications may be found in workplaces and homes. Point of use (i.e. nonclinical) applications include quality control testing, for example in the manufacture and packaging of food or pharmaceuticals, domestic chemicals, or testing of water quality.

All of these applications share the common requirement of a rapid turnaround and communication of results to guide decisions. In addition, point of use/care assays must be comparatively simple to perform, yet yield robust and reliable results. This combination of features is difficult to achieve and, as a consequence, such assays may provide only qualitative or semi-quantitative results. For example, many simple lateral flow type tests for pregnancy or stigmatized diseases such as HIV may provide a coarse “yes” or “no” result, to provide an indication as to whether further laboratory testing is required. However the results cannot in themselves inform clinical decisions.

The capability of conducting such competitive or sandwich assays in a point of care/use setting is in principle very desirable. Diagnosis without the need for samples to be referred to centralised testing laboratories can be particularly important in resource-limited settings, such as medical encounters in remote locations. The elimination of sometimes lengthy delays also has the potential to improve clinical outcomes more generally, by preventing patients from leaving a treatment conduit during the delay before results are available.

The requirement for an assay to be robust and reliable, and sufficiently simple to be performed in a point of use/care setting presents a significant barrier to implementation, and many types of assays are considered to be unsuitable for any type of point of care/use application. Efforts have been made to design assays which can be performed in a single step, by mixing reagents in a “one pot reaction” (also known as a “homogeneous” assay). However, even where this has been possible, the quality of data obtainable is comparatively poor. For example, enzyme-linked immunoassays (ELISAs) are sensitive to “noise” caused by components of the sample, or matrix. In order to obtain quantitative or semi-quantitative results, such assays must therefore include a complex series of reagent and rinse solution washing steps in order to remove the unwanted components. So called “matrix effects”, which are a barrier to point-of-care applications, are described for example by Chiu et al., Journal of Laboratory Automation, June 2010, 233-242. The influence of matrix effects on the quality of data and assay efficiency is also described for example by Saab et al., International Journal of High throughput Screening, 2010:1, 81-98, and Imbert, P. E. et al., Assay Drug Dev Technol., 2007, June; 5(3):363-72.

Simplifying such assays sufficiently to enable them to be performed at the point of care/use without the need for specialist training has therefore remained a challenge.

Accordingly, there remains a need for improved methods and apparatus for performing heterogeneous assays.

### SUMMARY OF THE INVENTION

According to an aspect of the invention there is provided an assay cassette for use in performing a heterogeneous assay, the assay cassette comprising

a fluid conduit extending between a sample inlet and a waste outlet; and
at least two chambers disposed in series along the fluid conduit, downstream of the sample inlet and upstream of the waste outlet;
each chamber comprising an immobilized analyte-specific probe, and adapted to enable acquisition of a respective measurement relating to the amount of immobilized analyte in each chamber, in use.

A sample to be assayed may be introduced into the fluid conduit through the sample inlet and thereafter caused to flow through each of the chambers in turn. In each chamber, an immobilized analyte-specific probe immobilizes a proportion of any of that analyte present in the sample, such that a measurement (e.g. an optical measurement) relating to the amount of immobilized analyte may be acquired from each of the chambers in turn.

Control over each step of an assay and the sequence in which steps of the assay take place is provided at least in part by the arrangement of the chambers in series along the fluid conduit. Thus, an assay cassette in accordance with the invention reduces amount of expertise and equipment required to perform an assay.

Moreover, a “multiplexed” assay (i.e. two or more assays conducted in two or more corresponding chambers) may be conducted without the need to automate the introduction of reagents from different points across the surface of a cassette or a chamber plate, e.g. by using expensive robotically moveable pipettes or other apparatus.

The assay cassette may comprise one or more reagent inlets in fluid communication with the fluid conduit, through which reagent may be introduced into the fluid conduit.

One or more reagent inlets may be in fluid communication with the fluid conduit at or upstream of the chamber immediately downstream of the sample inlet. One or more reagent inlets may be in fluid communication with the fluid conduit at or upstream of the sample inlet. The sample inlet may, in use, also function as a reagent inlet (for example the sample inlet may be used as an inlet for a washing fluid, to push/transport the sample through the fluid conduit).

One or more reagent inlets may be in fluid communication at or immediately upstream of a said chamber (i.e. a chamber in which the reagent is first required). In some embodiments, a reagent inlet is in fluid communication with the fluid conduit at or immediately upstream of each chamber.

A reagent inlet may communicate with the fluid conduit via a channel. A reagent inlet may communicate directly with the fluid conduit. For example, an inlet may be positioned in, or extend to, a wall of a said chamber.

Reagents, such as a washing fluid (e.g. water, a buffer solution) or a labelling fluid (e.g. a solution comprising a chromophore, or an analyte-specific probe labelled with a chromophore), may be introduced into the fluid conduit before, during or after introduction of sample into the fluid conduit and, accordingly, caused to flow through one or more of the chambers, before, during or substantially with the sample.

The cassette may comprise a reagent inlet for each reagent, or each type of reagent, required in an assay. Accordingly, no reagent inlet is required to be used for more than one reagent, or more than one type of reagent. This enables the sample and each reagent to be controlled completely independently, which simplifies automation of an assay. In addition, provision of independent inlets reduces the risk of cross-contamination of reagents.

The cassette may comprise one or more of;

a washing fluid inlet, for introducing a washing fluid such as water or a buffer solution into the fluid conduit;
a labelling fluid inlet, for introducing one or more labelling fluids each comprising a chemical label into the fluid conduit;
a conjugate fluid inlet, for introducing one or more conjugate fluids each comprising a chemical label bound to a control analyte, into the fluid conduit;
a triggering fluid inlet, for introducing a triggering fluid, one or more components of which combines with material already present in a chamber to trigger a measureable response (such as luminescence).

The cassette may comprise more than one of each type of reagent inlet. For example the cassette may comprise a labelling fluid inlet at or immediately upstream of each chamber.

In use, a reagent may be transferred from a reagent reservoir (external to, or in, the cassette) and introduced into the fluid conduit.

A reagent may be introduced into the fluid conduit along a channel. An end of a channel extending from a reagent reservoir may function as a reagent inlet.

A reagent may be introduced into the fluid conduit by displacing the reagent from a reagent reservoir, e.g. by compressing a reagent reservoir, or otherwise reducing its volume.

A reagent may be pumped into (and typically also along) the fluid conduit.

The analyte-specific probe of each chamber may be immobilized by binding, chemically or physically, to the walls of the chamber.

The analyte-specific probe of each chamber may be immobilized by binding to a solid support medium contained in the chamber.

A solid support medium contained in the chamber may increase the amount of immobilized analyte-specific probe in the chamber, for example by virtue of increased surface area, and so may enable more sensitive or accurate optical measurements to be acquired.

The solid support medium may comprise an absorbent or adsorbent material, such as a porous or molecular sieve material. The solid support medium may comprise a particulate material, such as beads (e.g. plastics beads, such as polystyrene beads) or a single bead. The solid support medium may comprise magnetic particulate material, for example magnetic micro- or nanobeads. Magnetic particulate material may comprise ferromagnetic or paramagnetic particles. Such particles may by provided with a paramagnetic or ferromagnetic coating. Such particles may comprise a metal oxide material, such as iron oxide, which may be impregnated in a plastics material.

The analyte-specific probe may be bound directly to a solid support medium, or may be bound to the solid support medium by a linker, such as a PEG linker.

The analyte-specific probe of each chamber may be immobilized by incorporation into a matrix formed by a solid support medium. For example, an analyte-specific probe may be dispersed or encapsulated in a polymer matrix of a polymeric/plastics solid support medium. Said medium may be porous.

The solid support medium may be retained in each chamber by way of a filter at the outlet from and/or inlet to the chamber.

The solid support medium may be retained in each chamber by virtue of the particle size of the solid support medium in comparison to the dimensions of the outlet/inlet of the chamber.

The solid support medium may be retained in each chamber, in use, by one or more magnets.

The analyte-specific probe and solid support medium may be provided within a soluble matrix material, as discussed further below.

The solid support medium may be retained in each chamber by virtue of its mass/inertia. Thus, in use, the rate of fluid flow along the fluid conduit may be insufficient to displace the solid support medium from its chamber.

The analyte-specific probe, and the solid support medium (where present) are preferably provided “pre-packaged” in each chamber during manufacture. This enables the materials to be prepared in bulk, which in turn enables the analyte-specific probe to be prepared economically and with high levels of quality control.

The analyte-specific probe may be selected for specificity with a particular analyte, which may be present in a given sample.

The cassette may comprise one or more reagents.

One or more reagents may be pre-packed on the cassette during manufacture, including or in addition to the immobilized analyte-specific probe(s).

In addition to an analyte-specific probe immobilized in each chamber, the cassette may further comprise one or more labelling fluids or one or more conjugate fluids.

The cassette may comprise one or more assay specific reagents (i.e. reagents specific to the or each assay for which the cassette is adapted, such as the analyte-specific probes, labelling fluid, conjugate fluid and, in some embodiments, triggering fluid).

Reagents suitable for use with one or more other types of assay, and thus another configuration of cassette, (e.g. washing fluid and, in some embodiments triggering fluid) may be provided externally.

It will be understood that a variety of different assays may be performed using a suitably configured assay cassette in accordance with the invention. Provision of all of the assay specific reagents in the cassette itself ensures that any assay may be performed using a common set of apparatus and reagents together with the respective assay cassette.

The cassette may comprise one or more reagent reservoirs for one or more assay specific reagents. The cassette may alternatively comprise one or more labelling materials or one or more conjugate materials, in solid form, downstream of a corresponding reagent inlet. The solid conjugate or labelling materials may be formulated to dissolve in contact with a fluid such as a washing fluid, so as to form a conjugate/labelling fluid in the cassette during use.

Accordingly, the cassette may comprise reagents (and in particular assay specific reagents) in solid or liquid form.

In certain embodiments, one or more reagents, such as assay specific reagents, are provided within a soluble matrix, which is capable of dissolving in the assay solvent so as to release the reagent(s) into solution in use.

An amount of the reagent may be provided in the form of a tablet (which may be placed in or adhered to a wall of a said chamber, or indeed provided as a coating to a wall or a chamber) or bead (which may be placed in a chamber) comprising a reagent-matrix composition, the reagent-matrix composition comprising one or more reagents in the soluble matrix (e.g. encapsulated, or mixed with or dispersed in the soluble matrix material).

The reagent-matrix composition (in the form of a tablet or bead, as the case may be) may be provided with a soluble coating, for example of a soluble coating material, capable of being eroded by the assay solvent. Thus, in use, the soluble coating may be eroded by fluids used to conduct the assay, so as to expose the reagent-matrix composition.

The invention extends in another aspect to a tablet or bead comprising a reagent-matrix composition, for use in performing an assay;

the reagent-matrix composition comprising a reagent (or reagents), in a soluble matrix, which is capable of dissolving in a reagent or a sample fluid used in the assay, so as to release the reagent(s) into solution in use;
the tablet or bead provided with a soluble coating, capable of being eroded by a reagent or a sample fluid used in the assay, so as to expose the reagent-matrix composition, in use.

The soluble matrix and the soluble coating may for example be water soluble.

The one or more reagents provided in solid form may be retained within a chamber of the cassette, by virtue of the size of the tablet or bead in comparison to the diameter of the fluid channel or any other inlets to our outlets from the chamber. For example, a tablet or bead may have a dimension (e.g. diameter) of the orders of millimetres (e.g. up to around 5 mm, or around 1, 2 or 3 mm), whereas the diameter of the fluid conduit is typically of the order of 100 microns to millimetres.

Advantageously, the matrix material is rapidly soluble in the solvent used for the assay (most typically water). A suitable matrix material may for example be a saccharide, such as a di-, tri- or oligosaccharide. Once such material is trehalose.

Rapid dissolution may be facilitated by the morphology of the reagent-matrix composition. For example, a relatively porous composition such as for a polycrystalline and/or loosely compressed tablet may to some extent promote capillary infiltration of a solvent and aid dissolution. Indeed dissolution may be promoted to some degree by disintegration of a tablet or bead comprising the reagent-matrix composition, on contact with a liquid, such as a sample.

Dissolution and/or subsequent mixing of the reagent with a fluid in the chamber may be facilitated by mechanical agitation, such as vibrating (e.g. using an eccentric motor) or using ultrasound. Such mechanical agitation may be applied by suitable apparatus forming part of a cassette reader.

A suitable coating material may for example be a water-soluble polymer. Suitable coating materials include for example materials used for enteric coatings, such as fatty acid esters (e.g. esters of aleurtic acid), alginates (e.g. sodium alginate), cellulose based polymers (such as cellulose acetate trimellitate) or a phthalate polymer (such as hydroxypropyl methylcellulose phthalate) or a soluble polyacrylate (such as poly(methacrylic acid-co-methyl methacrylate)), or other such coatings as will be known to those skilled in the art.

The soluble coating preferably dissolves relatively slowly in the solvent used for the assay (e.g. water), in comparison to the matrix material of the associated reagent-matrix composition. The soluble coating may be capable of substantially dissolving (e.g. 85%, 90%, 95% or approximately 100%) on contact with a liquid reagent such as a buffer, wash or sample solution, over a period of 5-10 or more times longer than the reagent-matrix composition. For example, the soluble coating may substantially dissolve on contact with a liquid reagent in a period of around 10-60 seconds, whereas the reagent-matrix composition may substantially dissolve on contact with the liquid reagent in a period of around 1-5 seconds.

A soluble coating may provide for increased shelf life of reagent(s) provided within the cassette, for example providing a barrier to moisture or oxygen, to protect the reagent-matrix composition.

In use, a soluble coating may delay dissolution of the reagent-matrix composition. Thus, such dissolution may be prevented whilst fluid (e.g. a sample) is flowing (e.g. whilst a chamber is filling). This may prevent reagent(s) from being carried out of the chamber whilst fluid is flowing along the fluid conduit—which would otherwise be to the detriment of the accuracy of an assay. Once a chamber is filled and fluid flow is stopped, the coating may be eroded away and the reagent(s) released into solution.

It will be within the ambit of a skilled person to select the matrix and coating materials, and the amount (e.g. the size, thickness, morphology etc.) of the associated sample-matrix composition and the thickness of the soluble coating, according to particular requirements.

It will be understood that the materials used for the soluble matrix and the soluble coating should be selected so as not to interfere with or in any way perturb the assay itself.

A tablet or bead for use in performing a competitive assay may for example comprise a conjugate-matrix composition, the conjugate-matrix composition comprising a labelled analyte (i.e. a chemical label bound to a control analyte), in a soluble matrix.

In some embodiments, a tablet or bead for use in performing an assay may comprise an immobilized analyte-specific probe, immobilized on a particulate solid support medium, in the soluble matrix. In preferred embodiments, the particulate solid support medium comprises magnetic particles (e.g. beads) to which the analyte-specific probe is immobilized.

Advantageously, therefore, one or more tablets or beads (coated or otherwise) may be provided within a chamber and retained therein for storage or transport, by virtue of its size and/or by adherence to a wall of the chamber. In use, the immobilized analyte-specific probe is released into contact with the analyte when the soluble matrix is dissolved.

A magnetic solid support in particular may be selectively retained in a chamber (by applying a magnetic field) when fluid is flowed along the fluid conduit (e.g. during a washing step) and selectively released to mix with reagent when fluid is not flowing.

By using a large tablet or bead comprising said magnetic solid support for storage and transport, and by selectively magnetically retaining the immobilized analyte-specific probes in a chamber, the dimensions of the channels and conduits of an assay cassette need not be sized so as to prevent the magnetic particles from leaving their respective chamber. This may facilitate use of channels, conduits and chambers of larger dimension, in turn allowing for use of larger amounts of sample and/or for faster assaying and/or greater sensitivity. Use of solid support having smaller particle size (such as magnetic microbeads, of the order of around 1 μm, 10 μm or 100 μm) provides for a larger surface area to which an analyte specific probe may be coated.

It will be understood that a tablet or bead need not comprise a reagent-matrix composition having reagent uniformly distributed therein. Indeed, in some circumstances it may be desirable for a reagent (for example analyte-specific probe immobilized on a solid support) to be concentrated in a part of the tablet or bead, such as the centre, so that the reagent is released over a shorter time period. A reagent may be encased, for example within a cavity, within a soluble matrix.

The invention also extends to an assay cassette for use in performing an assay, the assay cassette comprising;

a fluid conduit extending between a sample inlet and a waste outlet; and
a chamber disposed along the fluid conduit, downstream of the sample inlet and upstream of the waste outlet;
the chamber comprising a tablet or bead comprising a reagent-matrix composition, for use in performing an assay;
the reagent-matrix composition comprising a reagent or reagents, in a soluble matrix, which is capable of dissolving in the assay solvent so as to release the reagent(s) into solution in use
the tablet or bead provided with a soluble coating, capable of being eroded by the assay solvent, so as to expose the reagent-matrix composition, in use.

For example, an assay cassette for use in performing a competitive assay may comprise a tablet or bead comprising a conjugate-matrix composition (which may be located in or upstream of said chamber). An assay cassette may comprise a tablet or bead comprising an immobilized analyte-specific probe, immobilized on a particulate solid support medium, in a soluble matrix.

Each chamber may comprise one, or more than one, tablet or bead. A given chamber may comprise more than one type of tablet or bead.

For example, a tablet or bead comprising a conjugate-matrix composition may be provided in one chamber (i.e. for mixing) and a tablet or bead comprising an immobilized analyte specific-probe may be provided in another chamber in the fluid conduit downstream thereof.

A chamber may comprise both a tablet or bead comprising a conjugate-matrix composition and a tablet or bead comprising an immobilized analyte specific-probe.

A said chamber may comprise a tablet or bead comprising an immobilized analyte-specific probe which is specific to one type of analyte, and another said chamber may comprise a tablet or bead comprising an immobilized analyte-specific probe which is specific to another type of analyte.

The/each labelling or conjugate fluid may be provided with any suitable chemical label. A labelling fluid may comprise a chemical label bound (e.g. directly or via a linker) to an analyte probe. The labelling fluid may comprise a chemical label bound to an analyte-specific probe, to form a labelled analyte-specific probe. For example, a labelling fluid may comprise an enzyme label (an analyte specific probe) bound to a fluorescent or chemiluminescent substrate.

The labelled analyte-specific probe may be specific to the same type of analyte as the analyte-specific probe present in one of the chambers.

The cassette may comprise a labelling fluid for each chamber present on the cassette. Alternatively, the cassette may comprise a labelling fluid inlet for each chamber present on the cassette. In use a labelling fluid may be introduced into a respective chamber. In use, any immobilized analyte in the chamber may be labelled using the corresponding labelling fluid.

A labelling fluid may contact other analytes immobilized in other chambers (e.g. downstream of a respective chamber or labelling fluid inlet), but will only bind to and thereby label a specific analyte in the intended chamber or chambers in which that specific analyte has been immobilized.

Alternatively, a single labelling fluid may be provided, capable of labelling each of the analytes immobilized in the respective chambers in the cassette.

The chemical label may be bound to an analyte probe (e.g. an analyte-specific probe), so as to form a suitable labelled analyte probe. A labelled analyte-specific probe may be used in a labelling fluid or, in some embodiments, a conjugate fluid.

In the preparation of a conjugate fluid, a labelled analyte-specific probe may be attached (preferably covalently) to a control analyte. Alternatively, a chemical label may be bound directly to a control analyte.

A conjugate fluid may comprise more than one labelled analyte, so as to be capable of use in multiplexed competitive assays. Each labelled analyte may be labelled with the same, or with different, chemical labels.

The chemical label may be a chromophore. The chemical label may be a chemical moiety that reacts with another chemical substance (which may form part of a triggering fluid) to form a chromophore. For example, the chemical may comprise an enzyme capable of producing a coloured or fluorescent substance, or undergoing a luminescent reaction.

The chromophore may absorb one or more specific wavelengths of light. The chromophore may emit one or more specific wavelengths of light. The chromophore may be caused to emit one or more specific wavelengths of light by a physical, chemical or electrochemical stimulus.

The chromophore may be a luminescent or a phosphorescent moiety.

The chromophore may be a chemiluminescent moiety (such as a luminol or luminol derivative, or an acridinium or acridan ester) that may be caused to emit one or more specific wavelengths of light by chemical reaction with a triggering fluid. The chromophore may be released into solution during a reaction with a triggering fluid.

The chromophore may be an electroluminescent moiety, which may be caused to emit one or more specific wavelengths of light by application of a voltage, or consequent to a redox reaction caused by application of a voltage.

In a preferred embodiment, the chromophore comprises an acridinium ester moiety. The acridinium ester moiety may a temperature stabilised acridinium ester moiety, such as moieties (1) or (2):

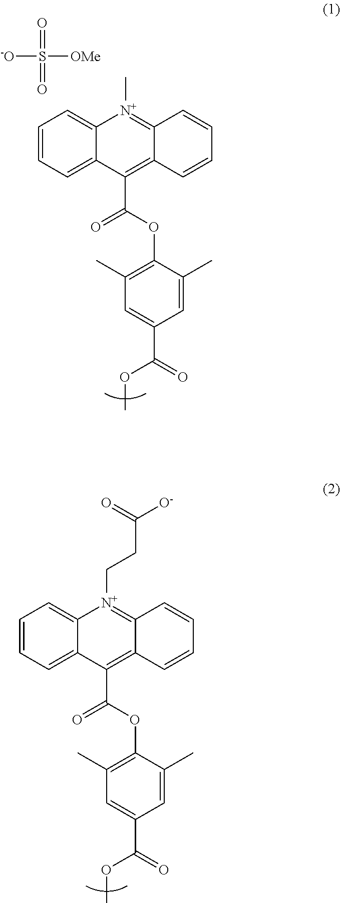

The chromophore may comprise an acridan ester, such as moiety (10):

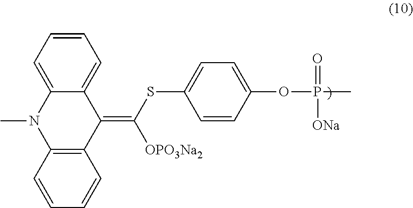

The moiety may be bound to an analyte probe (e.g. an analyte-specific probe), so as to form a suitable labelled analyte probe, for use in a labelling fluid or a conjugate fluid.

An acridinium ester moiety bound to an analyte probe or directly to a control analyte may be prepared from the corresponding N-hydroxysuccinimide (NHS) ester, (1a and 2a), by reaction with a nucleophilic species present on an analyte probe or a control analyte, by methods chamber known in the art.

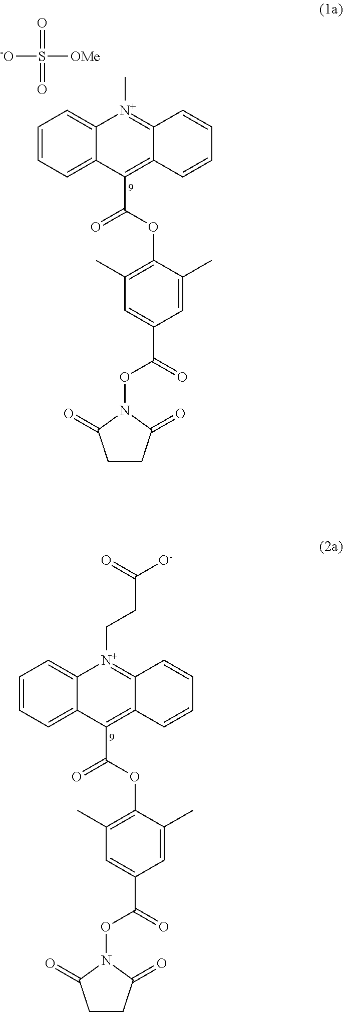

Acridinium ester moieties may be readily cleaved at the C9 position (marked with a numeral 9 in the structures (1) and (2) shown above), with a peroxide anion HO₂⁻, in accordance with the general reaction scheme I shown below:

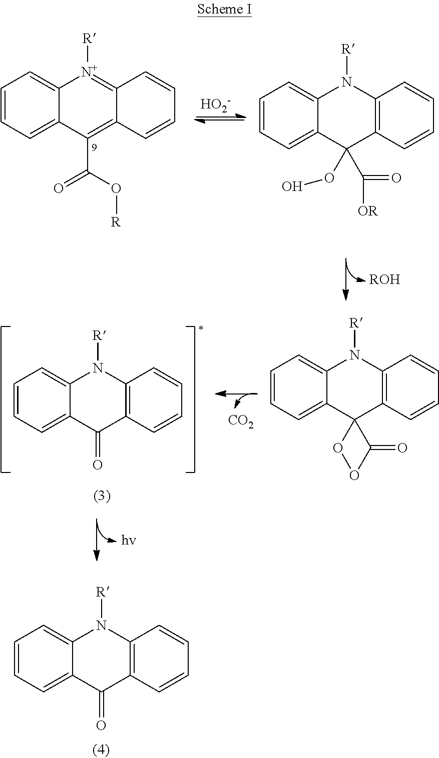

Following cleavage, an acridone (3) is released into solution in an excited state, which rapidly releases a photon hv as it relaxes to its ground state (4).

By virtue of the short timescale of their chemiluminescence (typically of the order of 5 seconds), which results in a high peak intensity of chemiluminescent light, acridinium esters advantageously provide for rapid and sensitive optical measurements,

The triggering fluid may comprise a peroxide solution, such as a mildly alkaline hydrogen peroxide solution, a urea peroxide solution or a benzyl peroxide solution.

Accordingly, the invention extends in another aspect to an assay cassette for use in performing a heterogeneous assay, the assay cassette comprising;

a fluid conduit extending between a sample inlet and a waste outlet; and
a chamber disposed along the fluid conduit, downstream of the sample inlet and upstream of the waste outlet;
a reagent reservoir, comprising an acridinium ester or an acridan ester labelling composition, in communication with the chamber;
the labelling composition comprising an acridinium/acridan ester labelled analyte probe (optionally an analyte-specific probe); and
the chamber comprising a immobilized analyte-specific probe, which may be specific to the same analyte as the labelling composition, and the chamber adapted to enable acquisition of a chemiluminescence measurement from acridinium/acridan ester bound to immobilized analyte in the chamber, in use.

The labelling composition may be in the form of a labelling solution, or may comprise a labelling material in solid form, which may be dissolved to form a labelling solution. The cassette may comprise more than one chamber, disposed in series along the fluid pathway.

The cassette may comprise a reagent reservoir.

The reagent reservoir may comprise a reagent in fluid form, pre-packaged in the cassette. The reagent reservoir may alternatively comprise a reagent material pre-packaged in the cassette, in solid form, formulated to dissolve to form a reagent fluid in use of the cassette.

The cassette may comprise a conduit extending between the reagent reservoir in the cassette and the fluid conduit. Alternatively, in use, reagent may be transferred between the regent reservoir in the cassette and the fluid conduit along an external conduit.

In use, a reagent may be pumped from the reagent reservoir in the cassette, along a conduit and through a reagent inlet.

The cassette may comprise a sealed reagent reservoir. Sealing a reagent reservoir minimises the amount of leakage, evaporation or absorption/adsorption which may occur over time (e.g. during storage of the cassette).

The sealed reagent reservoir may comprise a frangible member, for example a frangible membrane at or near an end of a conduit extending from the reservoir. A frangible member may be punctured, for example under the force of fluid pressure, in use of the cassette, so as to enable a reagent to be introduced into the fluid conduit.

Similarly, the entire fluid conduit may be sealed. i.e. The sample inlet, any reagent inlets, and the waste outlet may be sealed, for example by frangible members, and the seals only broken when the cassette is used.

A wall of a reagent reservoir may be frangible. For example, in use, a reagent reservoir may be pierced by an end of an external conduit.

Alternatively, or in addition, or more reagents may be introduced into the fluid conduit of the assay cassette in use, from one or more external reagent reservoirs. External reagent reservoirs may form part of an assay system with which the cassette is to be used.

The cassette may comprise a waste sink (or more than one such reservoir) in communication with (e.g. extending from) the waste outlet, which is preferably also enclosed. The waste sink may comprise an absorbent or adsorbent material.

The cassette may comprise one or more vents, for venting gas (e.g. air) displaced from the fluid conduit in use. The waste sink may comprise a vent.

The chambers of an enclosed fluid conduit each have a defined volume. The chambers may each have the same defined volume.

The enclosed volume of the portion or portions of the fluid conduit between adjacent chambers may be greater than the defined volume of the chamber immediately upstream thereof. The enclosed volume of the said portion/portions may be around twice, or three or more times the volume of the immediately upstream chamber.

Accordingly, an aliquot of a fluid (e.g. a reagent fluid) may be transported along the fluid conduit so as to enter each chamber in turn. Provision of sufficient enclosed volume of the fluid conduit between chambers enables the chamber to be flushed with an excess of a sample or reagent fluid, without the fluid passing into the next chamber downstream.

This arrangement enables use of a single labelling fluid (e.g. provided with a labelled analyte probe capable of binding to any of the analytes immobilized in any of the chambers) and/or a single triggering fluid. For example, an aliquot of labelling fluid may be pumped through each of the chambers in turn, followed by an aliquot of triggering fluid, so as to sequentially label any immobilized analyte in each chamber in turn. Chemiluminescence may then be triggered in, and a measurement of the chemiluminescence acquired from, each chamber in turn. This may be of particular advantage where a measurement must be acquired rapidly, e.g. in order to measure chemiluminescence having a short lifetime, such as in use of acridinium or acridan esters.

The cassette may be configured such that an aliquot of fluid may be passed along the fluid conduit so as to fill a single chamber at a time.

The portion(s) of the fluid conduit between chambers may be provided with sufficient volume by virtue of a combination of the length and diameter of the conduit. However, the greater the diameter of the conduit, the greater the amount of diffusion/mixing of different types of fluids may occur between successive aliquots introduced into the fluid conduit during an assay. Accordingly, the cassette may comprise one or more convoluted fluid conduit portions between chambers, so as to provide sufficient enclosed volume on a compact assay cassette.

The cassette may comprise one or more sensor arrangements along the fluid conduit for monitoring the progress of fluid along the fluid conduit. The cassette may comprise one or more sensor arrangements positioned along the fluid conduit between the chambers.

In use, information from the sensor arrangement(s) may be used to control how the assay is performed. For example, the/each sensor arrangement may be used to determine when to start and/or stop pumping each fluid or fluid type along the fluid conduit.

The one or more sensor arrangements may comprise electrodes extending into the fluid conduit. Thus, progress of fluid along the fluid conduit may be monitored by measuring conductivity. For example, progress of fluid along the fluid conduit may be monitored using step changes in conductivity, indicative of a change between a first type of fluid (e.g. a sample fluid) in the proximity of the sensor arrangement and a second type of fluid (e.g. a washing solution) in the proximity of the sensor arrangement.

The one or more sensor arrangements may comprise an optical sensor, operable to detect optical characteristics, or changes in optical characteristics, of fluid in the fluid conduit. For example, one or more fluids introduced into the fluid conduit may comprise an absorptive dye detectable using a said optical sensor.

The one or more sensor arrangements may comprise an indicator, such as a pH indicator (e.g. pH paper) which changes colour responsive to pH. Thus, the presence of a fluid in the region of the fluid conduit of each sensor arrangement having a particular pH, or a pH above or below a particular level, may be detected by way of a colour change of the indicator. The colour change may be detected by an optical sensor.

A said chamber may be adapted to enable acquisition of an optical measurement, such as a measurement of absorption, emission and/or scattering or light. A said chamber may be adapted to enable a spectroscopic measurement or measurements. Accordingly, a chamber may be provided with an optically transparent window. For example a face of a said chamber may be sealed with an optically transparent layer of material.

A said chamber may be adapted to enable acquisition of a transmission type optical measurement, and therefore may be adapted to enable light to be shone through the chamber from one side and detected on another side of the chamber, either across or through the cassette.

An optical measurement may be acquired at a specific wavelength, or across a specific range of wavelengths and therefore an optically transparent window may be optically transparent over at least the wavelength/wavelengths of the optical measurement. For example, a window may be transparent to IR, UV and/or visible light.

An optically transparent window/material may cause some scattering, refraction, reflection and/or absorption of the wavelength(s) of light to be measured, but it will be understood that a said window may be sufficiently transparent to enable a measurement to be acquired through the window.

A said chamber may be adapted to enable acquisition of an electrical measurement, such as a measurement of conductivity or resistivity. Accordingly, a chamber may be provided with one or more electrodes extending into the chamber.

The sample to be introduced into the fluid conduit via the sample inlet may be any suitable fluid sample. It may for example be a sample of fluid obtained from a human or animal subject, such as a whole blood, plasma, saliva, semen, sweat, serum, menses, amniotic fluid, tears, fluid containing material obtained from a tissue swab, urine, cerebrospinal fluid, mucous and the like.

The sample may, for instance, include materials obtained directly from a source, such as a sample of whole blood, as chamber as materials pretreated using techniques, such as filtration, precipitation, dilution, distillation, mixing, concentration, inactivation of interfering agents, etc.

Accordingly, the analyte to be detected can be any desired analyte and may include proteins, peptides, antibodies, nucleic acid, microorganisms (such as bacteria and viruses), chemical agents, toxins, pharmaceuticals, metabolites, cellular moieties and the like.

The analyte-specific probe may be any suitable agent that can bind specifically to the analyte to be detected. For example, if the analyte is a protein or peptide, the analyte-specific probe may be a receptor or antibody which is capable of specifically binding to the protein/peptide. Conversely an antibody may be bound by a protein/peptide which the antibody is designed to specifically bind to. Nucleic acids may be bound by other nucleic acids which are capable of specifically hybridising to the analyte nucleic acid. Microorganisms may be bound by antibodies which specifically bind to proteins on the surface of the microorganism.

Chemical agents, toxins, pharmaceuticals, metabolites may be bound by chemical moieties which are capable or reacting or binding to the aforementioned chemical analytes via appropriate bonding reactions, or affinities. Many types of binding techniques are chamber known to those of skill in the art.

Moreover, the analyte-specific probe may be an enzyme or an enzyme substrate. For example analytes such as glucose through chamber described enzymatic methodologies may be detected, for example the reaction product formed following the enzyme reacting with the glucose may be measured by using electrochemical, or optical detection techniques known to the skilled addressee.

The assay cassette may comprise a first chamber, comprising a first immobilized analyte-specific probe, and a second chamber, comprising a second analyte-specific probe.

The cassette may comprise more than two chambers, each comprising a different type of immobilized analyte specific probe.

At least two chambers may comprise the same type of immobilized analyte-specific probe. Thus, the cassette may be configured for an assay and a confirmatory assay to be conducted.

The assay cassette may be configured to perform a sandwich assay. Accordingly, the cassette may comprise one or more inlets to the fluid conduit for each of; washing fluid, labelling fluid and triggering fluid.

The assay cassette may be configured to perform a competitive assay. Accordingly, the cassette may comprise one or more inlets to the fluid conduit for each of; washing fluid and triggering fluid. The cassette may comprise one or more conjugate fluid inlets to the fluid conduit.

The cassette may further comprise a mixing portion of the fluid conduit upstream of the chambers, adapted to facilitate mixing of a conjugate fluid and a sample fluid, in use. The mixing portion may for example comprise a length of the fluid conduit sufficient to enable thorough mixing of the conjugate and sample fluids. The mixing portion may comprise one or more features, such as filters or weirs, to introduce turbulence into passing fluids and so to facilitate thorough mixing. The mixing portion may comprise a mixing chamber, disposed in the fluid conduit between the sample inlet and the most upstream chamber.

The invention extends in a further aspect to an assay cassette for use in performing a heterogeneous competitive assay, the assay cassette comprising;

a fluid conduit extending between a sample inlet and a waste outlet; and
a mixing chamber, disposed along the fluid conduit downstream of the sample inlet and upstream of the waste outlet; and in series with;
a chamber, disposed along the fluid conduit downstream of the mixing chamber and upstream of the waste outlet;
the chamber comprising an immobilized analyte-specific probe, and adapted to enable acquisition of a respective measurement relating to the amount of immobilized analyte in the chamber, in use.

The conjugate fluid inlet may be upstream of the mixing chamber and/or may be upstream of the sample inlet.

The cassette may comprise a mixing chamber filled with a predetermined amount of a conjugate fluid, into which a sample is introduced, in use.

The fluid conduit, and any channels extending to or from the fluid conduit, may be of capillary dimension. That is to say, the fluid conduit may be sized such that fluids, and in particular aqueous fluids, flow along the fluid conduit by capillary action. Devices in which the fluid is transferred predominantly by capillary action may be referred to as “microfluidic” devices.

The fluid conduit, and any channels, may comprise one or more fluid stop features, which function to prevent sample or other fluids from passing the fluid stop feature by capillary action alone. The fluid stop feature may for example comprise a hydrophobic material (such as a printable conductive or nonconductive material) or a hydrophobic surface feature (such as a surface region treated by laser ablation, surface scoring, and the like) which provides a hydrophilic/hydrophobic differential in the fluid conduit.

Accordingly, in use, control over the flow of fluid along the fluid conduit may be provided by one or more corresponding pumping arrangements, such as an electrostatic pumping arrangement, adapted to convey fluid past a respective fluid stop feature.

The fluid conduit may be configured, e.g. by virtue of the dimensions of the fluid conduit, such that fluid must be pumped in order to flow along the fluid conduit. Accordingly, in use, control over the rate and amount of each type of fluid flowing along the fluid conduit may be provided by one or more pumps (e.g. a pump associated with each fluid inlet). For example, the channels may have dimensions (height, width, diameter) of the order of 10⁻⁴-10⁻³ m (i.e. dimensions of the order of 100 microns to millimetres).

The cassette and the fluid conduit, the chambers and any channel(s), mixing chamber, reagent reservoir(s) and/or waste sink disposed therein may be formed in any manner of ways known to the skilled addressees, which may include photolithography, wet chemical etching, laser ablation, moulding (e.g. injection moulding), embossing and printing techniques.

The cassette and the fluid conduit and other features disposed therein may be formed by a sandwich of at least two layers. For example, the cassette may be formed from a substrate in which the fluid conduit and other features are defined, and a sealing layer (e.g. of optically transparent material) applied over the fluid channel and other features. The sample and other inlet(s)/outlet may be formed through the sealing layer.

The analyte-specific probes may be applied to the substrate before application of the sealing layer. Similarly, reagent(s) may be applied to any reagent reservoir(s) present, before application of the sealing layer.

Analyte-specific probes and/or reagents may be applied after assembly of the at least two layers (e.g. injected between the layers, and the injection ports subsequently sealed).

In some embodiments, the fluid conduit and other features are defined in part in each of two layers, which are joined together.

The cassette may be formed from three layers, or more than three layers.

The sealing layer may be flat. The sealing layer may define one or more features (such as optically transparent chamber walls).

Electrical features, such as electrodes and electrical leads thereto, may be applied to one said layer, during assembly, or may be sandwiched between layers. For example, electrical features may be deposited (e.g. by vapour deposition or printing) on a substrate.

The cassette may be formed of any suitable material, such as polycarbonate, polyester, polystyrene, PMMA, etc. and the/each layer may be formed of a single or plurality of material(s).

The two or more layers may be joined by any suitable method known in the art, for example by heat sealing, gluing, stapling and the like.

Openings or features in the top and/or bottom substrate may be designed to allow air to vent from the cartridge to allow filling with sample or co-locate with features in a reader device (as will be discussed hereinafter), which may facilitate with correct location of the cartridge in the reader.

According to a still further aspect of the invention there is provided an automated assay system, comprising;

an assay cassette according to other aspects of the invention and embodiments thereof, and
a reader device adapted to be coupled to the assay cassette, and comprising measurement apparatus for acquisition of a measurement relating to the amount of immobilized analyte in each of the chambers, in use.

The measurement apparatus may be adapted to acquire an optical measurement.

The measurement apparatus may comprise a photomultiplier tube or a charge coupled device (CCD), or any suitable light detection apparatus. The measurement apparatus may comprise an optical detector, such as a photocathode, or an optical lens and/or optical fibre. The measurement apparatus may comprise a light guide adapted to guide light between a chamber of the assay cassette and light detection apparatus such as a photomultiplier tube or CCD.

When the cassette is coupled to the reader device, the detector (and/or other components of the measurement apparatus) may be moveable in relation to the cassette, so that the detector can be positioned adjacent to each of the chambers in turn, so as to acquire an optical measurement from each chamber in turn.

The cassette may be moveable, and/or the detector may be moveable.

More preferably, the detector is configured to acquire an optical measurement from any of the chambers, without movement. For example, the photocathode of a photomultiplier tube may be sized to span all of the chambers of a cassette coupled to the reader device.

The reader device may comprise a shuttering arrangement, adapted to open and close an aperture adjacent to each said chamber. The shuttering arrangement may comprise, for example, one or more mechanical shutters, or one or more LCD shutters. The shuttering arrangement may comprise a moveable aperture.

The measurement apparatus may comprise a multi-anode photomultiplier tube. The measurement apparatus may comprise a light guide adapted to guide light between a chamber of the assay cassette to one or more selected anodes of the multi-anode photomultiplier tube. This arrangement may be used to direct and detect a signal only from a selected chamber. This arrangement may be used instead of, or optionally in addition to, a shuttering arrangement.

The measurement apparatus may comprise an optical emitter.

The detector may be configured to detect the same, or a different wavelength or range of wavelengths of light, to that emitted by the emitter.

The measurement apparatus may comprise more than one detector and/or more than one emitter. For example, the measurement apparatus may comprise more than one lens and/or more than one CCD, or light guide or length of optical fibre directing light to a detector, positioned to receive light from each respective chamber.

The measurement apparatus may comprise a triggering arrangement. For example, a chemical label may be electroluminescent, and light may be emitted consequent to a voltage being applied to fluid comprising the chemical label. An assay may include a step comprising an electrochemical reaction, induced by applying a voltage. Accordingly, the triggering arrangement may comprise means to apply a voltage, or pass a current, through a chamber. The triggering arrangement may be electrically connected to electrical connectors forming part of the cassette, when the cassette is coupled to the reader device.

The measurement apparatus may be adapted to acquire an electrical measurement. The measurement apparatus may be connectable to electrical connectors forming part of the cassette, e.g. electrical connectors in electrical contact with electrodes extending into the chambers.

A sample may be introduced into the fluid conduit of the cassette before the cassette is coupled to the reader device. The reader device may alternatively be provided with a sample port, or other sample receiving means, for receiving a sample and conveying the sample (e.g. by pumping, or by injection along a conduit) through the sample inlet of the cassette.

The reader device may be adapted to pump fluid, such as the sample, along the fluid conduit of the cassette. The reader device may comprise a pump, such as a piezo pump, a peristaltic pump, a syringe pump, an electrostatic pump or the like. The reader device preferably comprises a piezo pump or a syringe pump.

The reader device may comprise more than one pump.

The reader device may be adapted to introduce a reagent through a reagent fluid inlet of the cassette. Accordingly, the reader device may comprise means for transferring reagent from a reagent reservoir to a reagent inlet.

For example, the reader device may comprise a conduit (e.g. a flexible tube) extending from a reagent reservoir and which is connectable to a reagent inlet of the cassette, when the cassette is coupled to the reader device.

The reader device may comprise a reagent reservoir. The reader device may alternatively, or in addition, be connectable to an external reagent reservoir and/or to a reagent reservoir in the cassette.

The reader device may be adapted to open a sealed reagent inlet, for example by causing a frangible member across a reagent inlet to be pierced, when the cassette is coupled to the reader device. An end of a conduit extending from a reagent source may be used to open a sealed reagent inlet when the cassette is coupled to the reader device, in use.

The reader device may be adapted to open a sealed reagent reservoir, for example by causing frangible wall portion of a sealed reagent reservoir in the cassette to be pierced, when the cassette is coupled to the reader device.

The reader device may comprise a pump, configured to pump fluid from a reagent reservoir and to a reagent inlet, in use.

The pump may be configured to pump fluid along a conduit extending between the reagent reservoir and the reagent inlet.

In use, the pump may be used to pump fluid along the fluid conduit, or along a portion of the fluid conduit (e.g. the portion of the fluid conduit downstream of the reagent inlet and the waste outlet).

The pump may be any suitable type of fluid pump, such as a piezo pump, a peristaltic pump, a syringe pump, an electrostatic pump or the like. The pump is preferably a piezo pump or a syringe pump.

Alternatively, or in addition, the reader device may comprise a pumping arrangement, such as an electrostatic pump, arranged to cause fluid to pass a fluid stop feature within the fluid conduit or a conduit in communication therewith.

The reader device may comprise a pump adapted to pump reagent from a reagent reservoir in the cassette, along a conduit, to the reagent inlet (and in some embodiments along the fluid conduit). The conduit may form part of the cassette, or may form part of the reader device. The end or ends of a conduit forming part of the reader device maybe adapted to open the reagent reservoir and/or the reagent inlet.

The reagent reservoir may comprise a reagent, such as a washing fluid, a labelling fluid, a conjugate fluid or a triggering fluid.

The reader device may be adapted to introduce more than one reagent into more than one corresponding reagent inlet of the cassette.

The reader device may comprise more than one reagent reservoir and/or more than one pump (or pumping arrangement).

The reader device may comprise one or more non-assay specific reagent reservoirs (i.e. reservoirs for reagents which may be used with a number of assays of a general type, e.g. immunoassays, such as washing fluid or triggering fluid). This may enable the reader to be used with a number of different types of cassette, so as to perform a variety of different assays requiring non-assay specific reagents that are common to the variety of assays, by ensuring that the required reagent inlets are positioned to be connected to the source of the required reagents.

The reader device may comprise one or more sensor arrangements, for monitoring the progress of fluid along the fluid conduit. A said sensor arrangement may comprise an optical detector, for example operable to detect a step change in one or more frequencies of light absorbed, scattered or emitted by fluid in an adjacent region of the fluid conduit. A said sensor arrangement may comprise electrical circuitry connectable to electrodes extending into the fluid conduit and forming part of the cassette, the electrical circuitry operable to detect changes in the electrical properties of fluid in the fluid conduit in contact with the electrodes.

In use, the one or more sensor arrangements may be operable to generate fluid progress information, which may be used to control how the assay is performed.

The reader device may comprise means for regulating chamber temperature, such as a heater jacket or a thermoelectric device such as a Peltier device. Thus, the reader device may be operable to regulate the temperature of analyte immobilised in a chamber, in use, and thus to provide for controlled incubation periods.

The reader device may be adapted to apply a magnetic field to an assay cassette, capable of selectively retaining magnetic solid supports (e.g. beads) in a said chamber. The reader device may comprise one or more electromagnets or moveable magnets. The reader device may comprise a separate magnet or electromagnet associated with each said chamber of an assay cassette. The reader may comprise a single such (electro)magnet capable of applying a magnetic field across an entire assay cassette.

The reader device may be adapted to mechanically agitate material present in a chamber of an assay cassette which is coupled to the reader device. The reader device may for example comprise an eccentric motor, capable of transmitting vibration to the cassette when actuated. The reader device may comprise an ultrasonic generator (for example a piezoelectric transducer) capable of transmitting ultrasonic energy to the cassette when actuated.

The assay system may comprise a processing resource, configured to control the performance of an assay, using the assay system. The processing resource may be configured to control operation of the reader device, such as the timing of operation of the measuring apparatus, pumps or pumping arrangements (where present), means for regulating temperature and so forth. The processing resource may be configured to receive and respond to fluid progress information.

The processing resource may be configured to receive measurement data from the measurement apparatus, and process the measurement data. The processing resource may be configured to output processed measurement data. The processed measurement data is preferably quantitative or semi-quantitative. The processed measurement data may for example relate to the concentration of an analyte, or the relative concentration of an analyte to another component present in a sample (e.g. another analyte).

A cassette may be provided with assay instruction data, e.g. provided by way of bar code or smart code printed on the cassette, or a radiofrequency identification (RFID) tag. The reader device may be provided with an assay data reader (e.g. a scanning laser reader, or a radio frequency transceiver for interrogating an RFID tag), such that assay instruction data may be obtained from the cassette. The processing resource may be configured to receive assay instruction data and execute a corresponding series of controls over the abovementioned components of the reader device, so as to carry out the particular assay for which the cassette coupled to the reader is intended.

The processing resource may take the form of a computer processor, or more than one processor. The reader device may comprise the processing resource, or the processing resource may be external to and connectable to the reader device (e.g. a computer running suitable control software). The processing resource may comprise one or more processors working together and, for example, may comprise a computer processor of an external computing device connected (e.g. directly, or across a network) to a processor in the reader device.

The assay system may comprise a user interface, or more than one user interface. For example, reader device may comprise a user interface (for example one or more buttons, or indicators) and be connectable to a computing device having a user interface (e.g. a keyboard, screen and the like).

The cassette may be coupled to the reader device by inserting the cassette into a slot in the reader device.

It is to be appreciated that the assay cassettes and assay systems of the present invention may be applied in the human health area, including large and growing IVD markets (e.g. cancer, cardiology, diabetes and infectious disease), C-reactive protein assays (which can be used for measuring the inflammatory state in polymyalgia rheumatica), and to the veterinary diagnostics sector (e.g. testing of thyroid function).

The assay cassettes and systems of the present invention may also be used to test drugs and drug action. However, the system may also be applied in environmental settings where it is desirable to detect, for example toxic agents or infectious agents such as bacteria or viruses, for example to test potability of water. Thus, samples from rivers or lakes or swabs from solid surfaces may be taken in order to obtain a fluid sample for providing to the cassette. The assay systems may also be utilised for veterinary applications for laboratory, point of care and in the field testing. Essentially any assay in which a sample can be provided in a fluid form may be utilised in the present invention.

According to another aspect of the invention, there is provided a method of performing an assay, comprising:

introducing a sample fluid into a fluid conduit through a sample inlet to the fluid conduit; causing the sample fluid to pass along the fluid conduit, and into a chamber disposed in the fluid conduit;
using immobilized analyte-specific probes present in the chamber to immobilize at least a portion of any of the specific analyte present in the sample fluid within the chamber; and
acquiring a measurement of the chamber, relating to the amount of immobilized analyte therein.

The method may comprise causing the sample fluid to pass along the fluid conduit, and into each of at least two chambers disposed in series along the fluid conduit;

using immobilized analyte-specific probes present in each of the chambers to immobilize at least a portion of any of the specific analyte or analytes which are present in the sample fluid within the respective chambers; and
acquiring a measurement of each of the chambers, relating to the amount of immobilized analyte therein.

The method may comprise introducing a labelling fluid, comprising an acridinium ester or acridan ester labelled probe into a said chamber and immobilizing the acridinium ester or acridan ester labelled probe on at least a portion of any of the specific analyte which has been immobilized in the chamber;

triggering a chemiluminescent reaction of the immobilised acridinium/acridan ester; and acquiring a chemiluminescence measurement of the chamber, relating to the amount of immobilized analyte therein.

The method may comprise providing a conjugate fluid, comprising a labelled analyte, in the fluid conduit;

mixing the sample and conjugate fluids in a mixing chamber positioned in the fluid conduit downstream of the sample inlet;
causing the mixed fluid to pass along the fluid conduit and into a chamber, disposed along the fluid conduit in series with and downstream of the mixing chamber;
using immobilized analyte-specific probe present in the chamber to immobilize at least a portion of the labelled analyte and of any unlabelled analyte present in the mixed fluid within the chamber; and
acquiring a measurement of the chamber, relating to the amount of immobilized labelled analyte therein.

The conjugate fluid may be provided by introducing the conjugate fluid into the fluid conduit through a conjugate fluid inlet.

The method may comprise providing a tablet or bead in accordance with other aspects, in the fluid conduit (e.g. in a mixing chamber); the tablet or bead comprising a conjugate-matrix composition, the conjugate-matrix composition comprising a labelled analyte in a soluble matrix. The conjugate fluid may be provided by dissolving the soluble matrix (e.g. in a sample fluid) so as to release the labelled analyte.

The method may comprise providing a tablet or bead in accordance with other aspects, in the fluid conduit; the tablet or bead comprising; a reagent-matrix composition, comprising a reagent (or reagents), in a soluble matrix; and dissolving the soluble matrix (in a reagent fluid or sample fluid) so as to release the reagent (or reagents).

The tablet or bead may be provided with a soluble coating. The method may comprise eroding the soluble coating with a reagent fluid or a sample fluid, so as to expose the reagent-matrix composition.

The tablet or bead may comprise a conjugate-matrix composition. The method may comprise forming a conjugate fluid by dissolving a soluble matrix and thereby releasing a conjugate therefrom.

The tablet or bead may comprise an immobilized analyte-specific probe, immobilized on a particulate solid support medium, in a soluble matrix. The method may comprise releasing said immobilized analyte-specific probe by dissolving the soluble matrix.

The solid support may comprise magnetic particles. The method may comprise retaining the magnetic particles in a chamber, for example when a reagent or other fluid is flowing along the fluid conduit and/or into or through the chamber, by applying a magnetic field.

The method may comprise use of the cassette, or the assay system, of the other aspects of the invention.

The method may comprise acquiring the sample fluid, for example from a subject, and performing the assay, within a single subject/problem encounter. That is to say, the method may comprise acquiring the sample fluid and performing the assay at the point of care, or at the point of use, as the case may be.

The method may comprise introducing one or more reagents into the fluid conduit. One or more reagents may be introduced through the sample inlet. One or more reagents may be introduced through a reagent fluid inlet in communication with the fluid conduit, or through one or more corresponding reaction fluid inlets.

The method may comprise mixing a reagent, such as a conjugate fluid, with the sample fluid.

Mixing may be conducted before introduction of the resulting mixed fluid into the fluid conduit, mixing may be conducted during or after introduction of the fluids into the fluid conduit. For example, mixing may be conducted along a portion of the fluid conduit, and/or in a mixing chamber disposed along the fluid conduit.

The method may comprise introducing one or more of the following reagents into the fluid conduit;

a washing fluid such as water or a buffer solution;
a labelling fluid, comprising a chemical label;
a conjugate fluid, comprising a chemical label bound to a control analyte;
a triggering fluid, one or more components of which combines with material, e.g. a chemical label, to trigger a measureable response.

Accordingly, the method may comprise one or steps of each of:

washing, so as to wash excess or non-immobilized material out of a chamber;
immobilizing, so as to immobilise analyte (labelled, from a conjugate fluid, and/or unlabelled, from the sample) or to immobilize a labelled analyte specific probe;
triggering, so as to trigger a measurable response from material (e.g. a chemical label) present in a chamber.

One or more steps of the assay, in particular where mixing of reagents is required or where washing of immobilized species is required, may be facilitated by mechanically agitating the cassette or a chamber thereof. The method may at various stages comprise for example vibrating (e.g. using an electric motor) or ultrasonically agitating, the cassette.

Non-immobilized components might include, for example, excess analyte, or other types of analyte which an analyte-specific probe in a given chamber is not adapted to immobilize, or might include excess labelled analyte-specific probe.

One or more said steps of immobilizing may comprise incubating material in a chamber, by maintaining the chamber at a predetermined temperature (e.g. body temperature) for a predetermined period of time.

A measurement may be acquired from a chamber during and/or after triggering. Acquisition may in some embodiments commence before triggering.

The method may comprise triggering a measureable response from material present in a first chamber, acquiring a measurement from the first chamber, and subsequently triggering a measurable response from a second chamber and acquiring a measurement from the second chamber. The method may alternatively comprise triggering a measurable response from a first and a second chamber, and subsequently acquiring a measurement from the first and second chambers.

The method may comprise pumping one or more fluids along, and/or into, the fluid conduit. Pumping provides for control over the progress of fluid along the fluid conduit.

The method may comprise coupling a cassette to a reader device.

The sample may be introduced into the fluid conduit prior to the cassette being coupled to the reader device, or after the cassette has been coupled to the reader device.

Coupling the cassette to the reader device may comprise placing one or more reagent reservoirs in fluid communication with the fluid conduit. Coupling the cassette to the reader device may comprise opening a sealed sample inlet and/or one or more sealed reagent inlets (for example by piercing one or more corresponding frangible members). Coupling the cassette to the reader device may comprise opening one or more sealed reagent reservoirs in the cassette (for example by piercing a frangible portion of a wall defining a said reagent reservoir).

The method may comprise triggering a measurable response and acquiring a measurement of a first chamber, and subsequently triggering a measurable response and acquiring a measurement of a second chamber.

The measureable responses may be triggered in the first and second chambers using a single aliquot of triggering fluid. In embodiments where the volume of the portion of the fluid conduit between the first and second chambers is greater than the volume of the chambers, the first chamber may be flushed with an excess of triggering fluid before the leading end of the aliquot of fluid reaches the second chamber. The second chamber may then also be flushed with an excess of the triggering fluid, once the measurement has been acquired from first chamber.

The method may comprise causing the sample fluid to pass along the fluid conduit and into each of the at least two chambers, and subsequently passing one or more reagent fluids along the fluid pathway.

The method may comprise passing an aliquot of sample fluid, or a mixture of a sample fluid and a conjugate fluid, along the fluid conduit (for example, conveyed by an aliquot of a reagent fluid such as a washing fluid, subsequently introduced into the fluid pathway, optionally via the sample inlet), and filling one chamber at a time.

The method may comprise introducing a series of fluid aliquots into the fluid conduit. The series of aliquots may then be pumped or otherwise conveyed along the fluid conduit and through each of the chambers in turn, in the sequence as required by the particular assay.

For example, the method may comprise introducing into the fluid conduit a sample fluid, or an aliquot of sample fluid, followed by an aliquot of a labelling fluid, followed by an aliquot of a triggering fluid (optionally also introducing an aliquot of washing fluid after the sample fluid and/or labelling fluid and/or triggering fluid).

The method may comprise monitoring the progress of fluid along the fluid conduit, for example using one or more sensor arrangements disposed along the fluid conduit.

The method may comprise introducing a labelling fluid, or a conjugate fluid, comprising an acridinium or acridan ester, into the fluid conduit. The method may comprise introducing a triggering fluid comprising a peroxide species, such as an alkaline hydrogen peroxide solution, into the fluid pathway. Accordingly, the method may comprise acquiring a measurement of chemiluminescence emitted by an acridone compound, from a said chamber.

The method may comprise coupling a first cassette to the reader device, and performing a first assay or multiplexed assay; and

coupling a second cassette reader device, and performing a second assay or multiplexed assay.

The first and second cassettes may be the same or may be different.

Further preferred and optional features of each aspect of the invention correspond to preferred and optional features of any other aspect of the invention.

### DESCRIPTION OF THE DRAWINGS

Example embodiments of the invention will now be described with reference to the following figures, in which:

FIG. 1 shows an exploded view of an assay cassette in accordance with the invention;

FIG. 2 shows a perspective view of components of an assay system in accordance with the invention;

FIG. 3 shows a perspective view of an alternative embodiment of an assay cassette, adjacent to a shutter arrangement and measurement apparatus;

FIG. 4 shows a schematic view of the cassette of FIGS. 1 and 2 in a reader device;

FIG. 5 shows a (a) front and (b) rear exploded view of a multi-anode photomultiplier tube for use with an assay cassette.

FIG. 6(a)-(g) schematically show the steps of a multiplexed sandwich assay performed using the cassette of FIG. 1;

FIG. 7 shows a schematic diagram of another embodiment of an assay cassette adapted for use in performing a multiplexed competitive assay;

FIG. 8 shows a still further embodiment of an assay cassette in accordance with the invention;

FIG. 9 shows another embodiment of an assay cassette in accordance with the invention, for use in performing a multiplexed competitive assay;

FIG. 10(a)-(f) schematically shows the steps of a competitive assay;

FIG. 11 shows CLISA luminescence measurements acquired of Salmonella enteritidis from a range of 25 g food samples;

FIG. 12 shows CLISA luminescence measurements acquired of Salmonella typhimurium from a range of 25 g food samples;

FIG. 13 shows CLISA luminescence measurements acquired of Salmonella Goldcoast from a range of 25 g food samples;

FIG. 14 shows CLISA luminescence measurements acquired of Salmonella Seftenberg from a range of 25 g food samples; and

FIG. 15 shows CLISA luminescence measurements acquired of Salmonella Dublin from a range of 25 g food samples;

FIGS. 16 and 17 show luminescence measurements acquired on protein-coated magnetic microbeads (using 0, 25, 50, 100, 200 and 400 μg/ml of T4-BSA or anti-T4 and detected with 40 pmol/ml anti-T4-Acr ester or T4-HRP respectively); and

FIGS. 18 and 19 show luminescence measurements acquired using a competitive assay conducted, respectively, on T4-BSA or Anti-T4, which wer subsequently incubated with 40 pmol/ml anti-T4-Acr ester or T4-HRP respectively and increasing concentrations of T4-BSA. Data were acquired over the range of approximately 1 to 100 pmol/ml T4-BSA (FIG. 18) and anti-T4 coated beads showed inhibition over the range of approximately 1 to 400 pmol/ml (FIG. 19).

### DETAILED DESCRIPTION OF EXAMPLE EMBODIMENTS

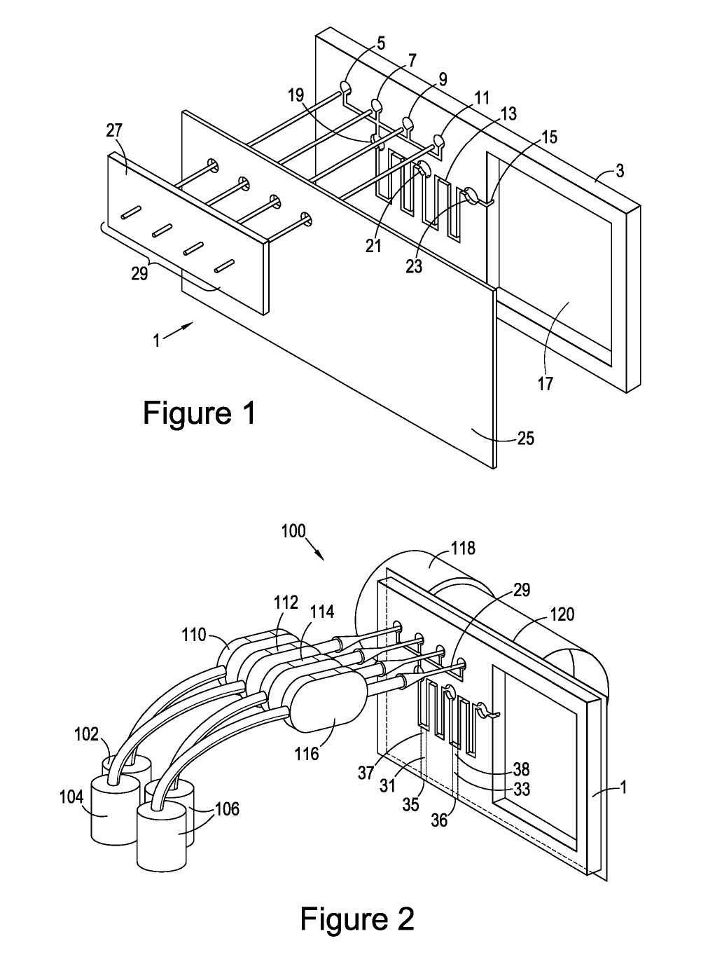

FIG. 1 shows an exploded view of an assay cassette 1 in accordance with the invention. The cassette is constructed from a substrate 3, formed from a low-protein binding clear plastics material (e.g. polypropylene). Moulded into the face of the substrate is a sample/wash inlet 5 and a series of reagent inlets 7, 9, 11.

A fluid conduit 13 extends between the sample inlet 5 and a waste outlet 15. The waste outlet opens into a waste sink 17, which is moulded into the substrate 3.

Chambers 19, 21 and 23 are moulded into the substrate 3, and are disposed in series along the fluid conduit. The substrate is composed of an optically transparent material and the thickness of the base of each of the chambers is sufficiently small as to enable optical measurements to be acquired through the substrate, as described in further detail below. Although three chambers are shown, the cassette may, in alternative embodiments, include other numbers of chambers.

Prior to use of the cassette, the chambers are each filled with polystyrene beads (not shown), each coated with an analyte-specific antibody (i.e. a probe). The beads are larger than the opening of the fluid conduit to the chamber and so are retained within the chambers in use.

The fluid conduit 13 (including the bead-containing chambers 19, 21, 23) and the waste sink 17 are enclosed by a polypropylene covering sheet 25 and the inlets 5, 7, 9, 11 are sealed by a silicone layer 27. Thus, prior to use of the cassette, the fluid conduit is entirely sealed.

In FIG. 1, the position of needles 29 for introducing reagents into the fluid conduit in use, is illustrated for explanatory purposes.

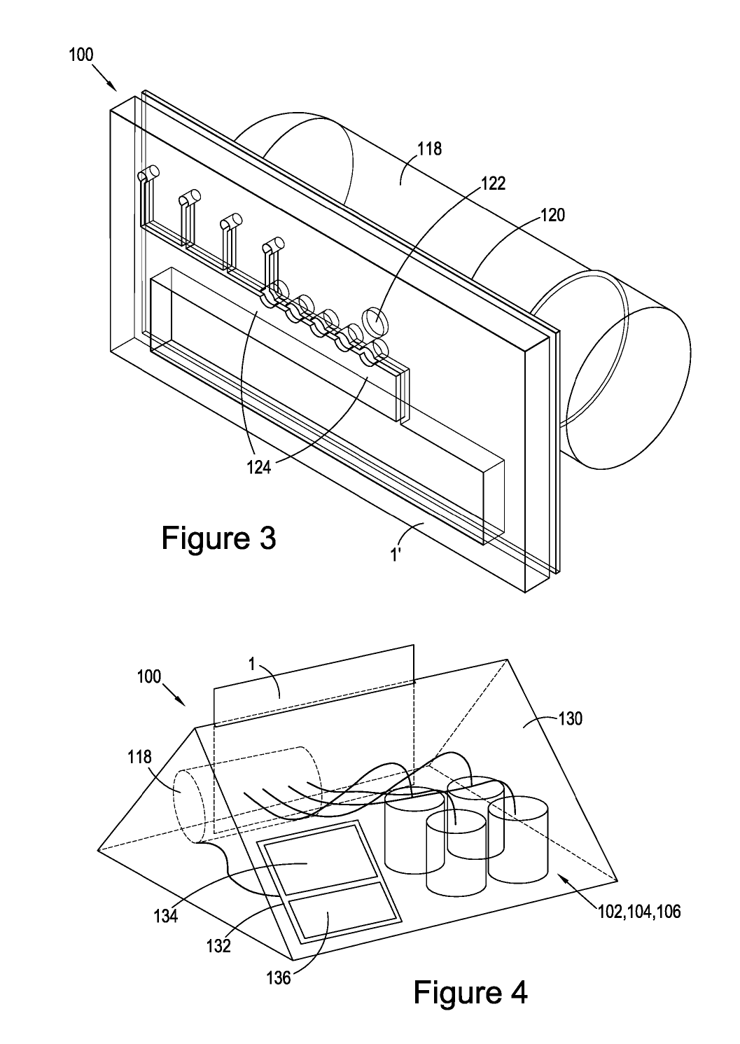

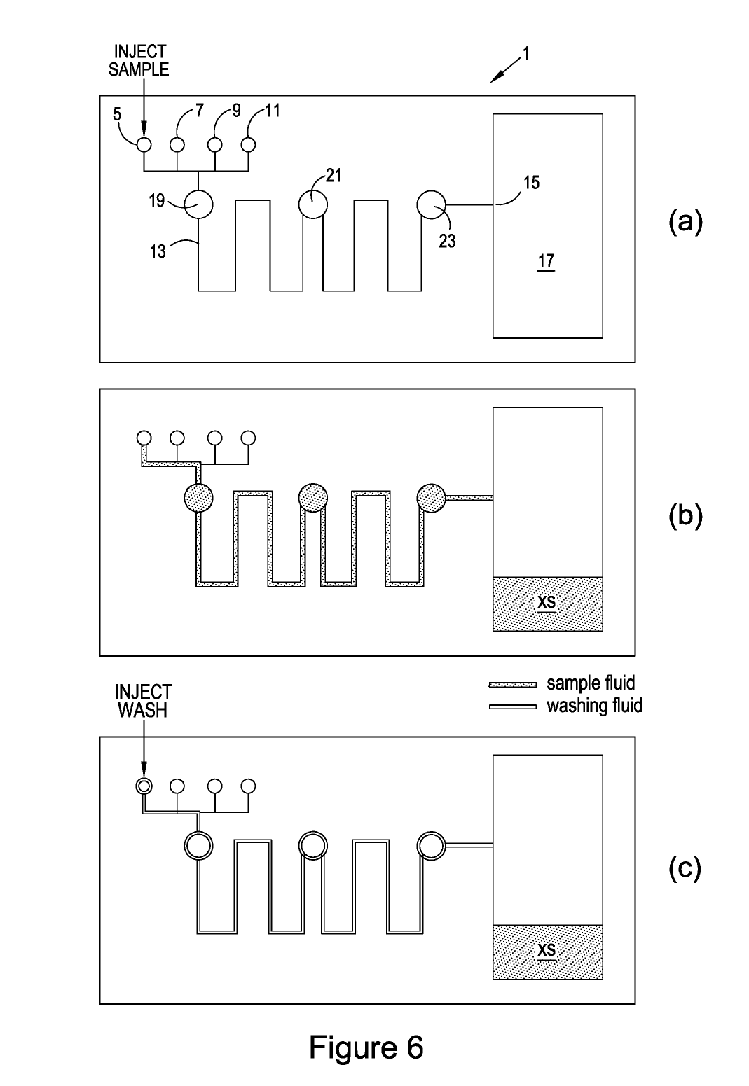

FIG. 2 shows the cassette 1 and other component parts of an assay system 100 in accordance with the invention. The other parts are normally packaged within a reader device (shown in FIG. 4).

The assay system 100 comprises reagent reservoirs 102, 104 and 106, for storing a washing fluid, a labelling fluid and a triggering fluid, respectively. The reagent reservoirs are connected by conduits 108, to respective retractable injection needles 29. The conduits extend through piezoelectric pumps 110, 112, 114 and 116, which can operate to independently control delivery of reagent from each of the reservoirs. The covering sheet 25 and silicone layer 27 of the cassette 1 are omitted for clarity.

Adjacent to the cassette is a photomultiplier tube (PMT) 118. The PMT is of a side on, reflectance configuration and includes a photocathode (not shown) which extends across an area adjacent to all three chambers 19, 21, 23. Between the cassette 1 and the PMT 118, is an LCD shutter 120.

Also visible in FIG. 2 are pairs of electrical conductors 31, 33 (copper strips) which are sealed between the substrate 3 and the covering sheet 25. The conductors extend from terminal ends 35, 36 to electrode ends 37, 38 in fluid contact with the fluid conduit 13. In use, the terminal ends 35, 36 are connected to a suitable sensor arrangement and an alternating current may be applied, in order to measure AC conductivity across the electrode ends 37, 38. Step changes in conductivity are characteristic of differences between each of the reagent fluids being present in the region of the fluid conduit between the electrode ends and this enables the conductors 31, 33 (in conjunction with the sensor arrangement) to be used to monitor the progress of the fluids along the fluid conduit, in use.

In alternative embodiments, the electrical conductors and electrodes are omitted and progress of the fluids (and the timing of the acquisition of optical measurements) is determined based on the volume and rate of fluid pumped through the fluid conduit. Optionally, in order to ensure that each measurement is not contaminated with light from an adjacent sample chamber, shutter timing may be controlled so that the shutter is opened shortly after the start of, and closed shortly before the end of, the triggering of the chemiluminescence reaction in each chamber i.e. If there are N chambers, a shutter may be opened adjacent to each of them for slightly less than 1/N of the total duration of the assay. Another alternative would be to deliver the trigger reagents very rapidly so that all of the chambers are emitting simultaneously. The LCD shutters would then be used to switch rapidly and repeatedly between the chambers sampling each of them for slightly less than 1/N of the total duration of the assay.

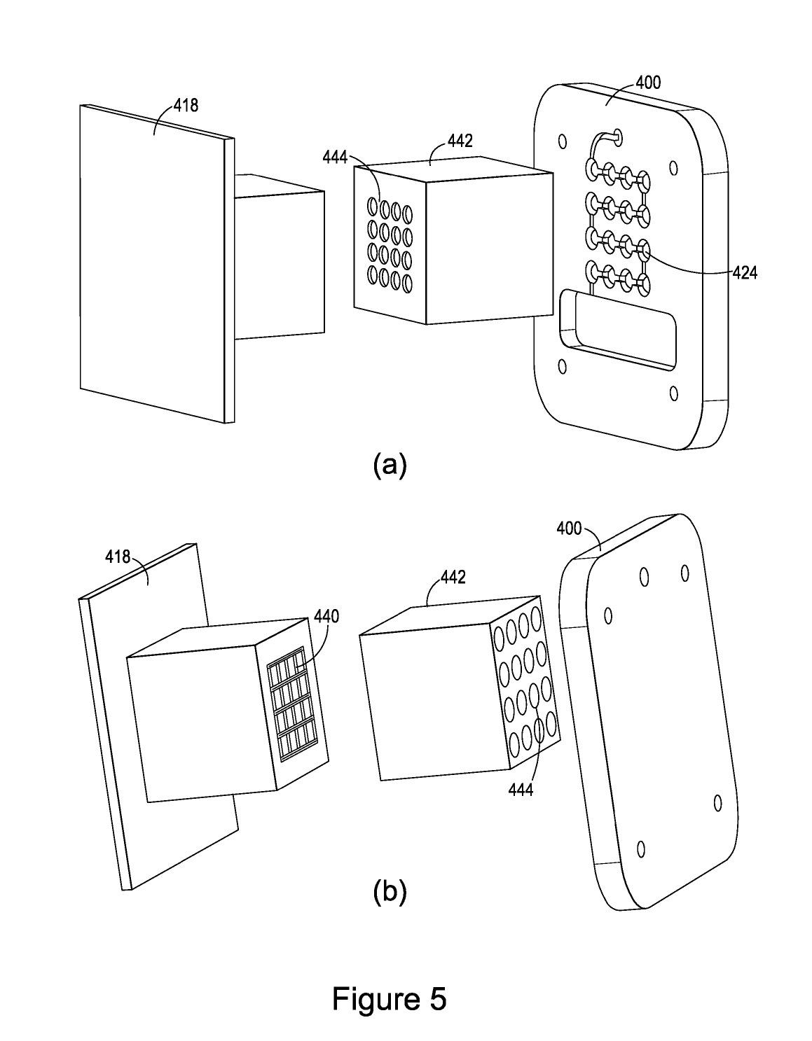

As can be more clearly seen in FIG. 3, which shows a cassette 1′ adjacent to a PMT 118 (other features omitted for clarity), in use, the LCD shutter 120 can be controlled so as to control the position of the aperture 122 in relation to the chambers 124. The accuracy of optical measurement from a chamber which has been triggered is thereby optimised, and the amount of residual light reaching the photocathode of the PMT from a previously triggered chamber is minimized.

The component parts of the assay system 100 shown in FIGS. 1 and 2 are housed within a reader device 130, which is shown schematically in FIG. 4. The reader device houses the reagent reservoirs 102, 104, 106 and the pumps, conduits and needles described above. The cassette 1 is shown having been inserted into a slot within the reader device 130.

The reader device further comprises a computing device 132 (comprising a computer processor running control software) connected to the PMT 118, and comprising a display 134 and a user interface 136. The computing device is also in communication with the LCD shutter 120, the conductors 31, 33 and the pumps 110, 112, 114, 116, however these features and the connections are omitted FIG. 4, for clarity.

The computing device is operable to control the operation of the pumps, shutter and the PMT, so as to control how the assay is performed. The computing device receives measurement data from the PMT, and is configured to calculate and output, to the display, quantitative measurement data relating to analyte concentrations. Operation of the reader device, for example to start an assay, process data (in some embodiments) and conduct operations such as calibration, cleaning and the like, is effected by way of the user interface.

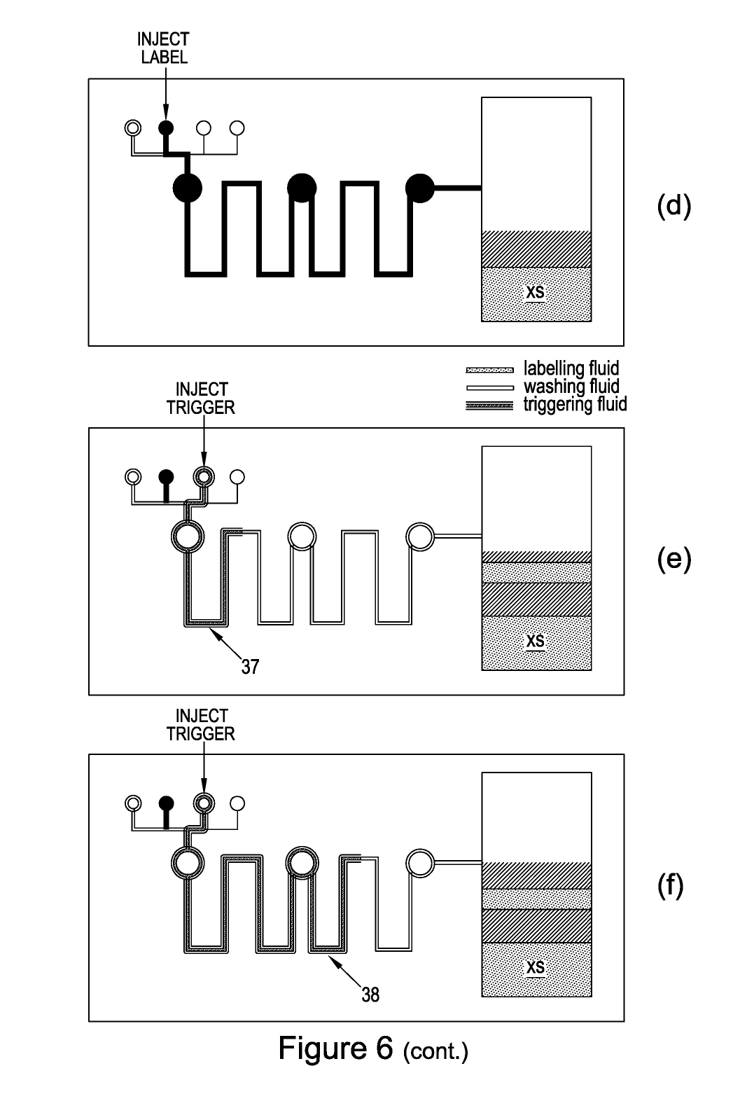

FIG. 5 shows an alternative embodiment of measurement apparatus, comprising a multi-anode PMT 418. In the embodiment shown, the multi-anode PMT has 16 anodes 440, and is adapted for use with a 16-chamber assay cassette 400. A cassette reader device comprising the PMT 418 is also provided with a light guide block 442 having, in the embodiment shown, 16 channels 444 corresponding to the array of 16 anodes 440. The channels 444 can be aligned, in use with the chambers 424 of the cassette 400. Accordingly, light from each chamber is directed to a specific anode of the multi-anode PMT.

Use of the cassette 1 and the system 100 to perform a multiplexed sandwich assay will now be described.

The cassette may be configured to perform any suitable assay as known to one skilled in the art. In a preferred embodiment, the cassette is configured to conduct a thyroid function test, for levels of the thyroid-stimulating hormone (TSH, thyrotropin) and thyroxine (T4), and triiodothyronine (T3) in a patient's blood sample. Antibodies for each hormone, suitable for use as analyte-specific probes, may be raised against bacteria such as Salmonella, E-coli. Antibodies specific for each of the three hormones may be chemically immobilized on polystyrene beads using conventional methodology (for example as described by H. G. Hilton and P. Parham, Tissue Antigens, 2013, 81, 212-220), and placed in each of the chambers 19, 21 and 23. Similarly, three further antibodies specific to each of the three hormones may be labelled with acridinium ester moieties (1) and made up into suitable labelling solutions.

In an alternative embodiment (not shown) an assay cassette may be configured to test for C-Reactive Protein (CRP). C-reactive protein (CRP) is an acute-phase marker, measurements of which can be used for measuring the inflammatory state in polymyalgia rheumatica, and is also a useful prognostic indicator in patients with acute coronary syndrome, as elevated CRP levels are independent predictors of cardiac death, acute myocardial infarction and chronic heart failure. Normal levels are in the high ng/ml to low μg/ml range. In any acute disease process, the human liver produces CRP and levels in blood can increase to hundreds of μg/ml. CRP has presented a problem for prior art POC analytic systems because of the wide dynamic range of the antigen to be measured (>105-fold). However, following serial dilution, a multiplexed assay cassette in accordance with the invention may be used for the analysis of a sample. Chemically derivatised beads having monoclonal anti-CRP antibody bound to their outer surface were mounted in each serial reaction chamber together with a detector-antibody solution (DMAE-labeled monoclonal anti-CRP having a different epitope specificity than that on the beads). Following a pumped wash cycle with 0.2M Tris/NaCl buffer, pH8.0, luminescence was activated with alkaline hydrogen peroxide, pH10.5.

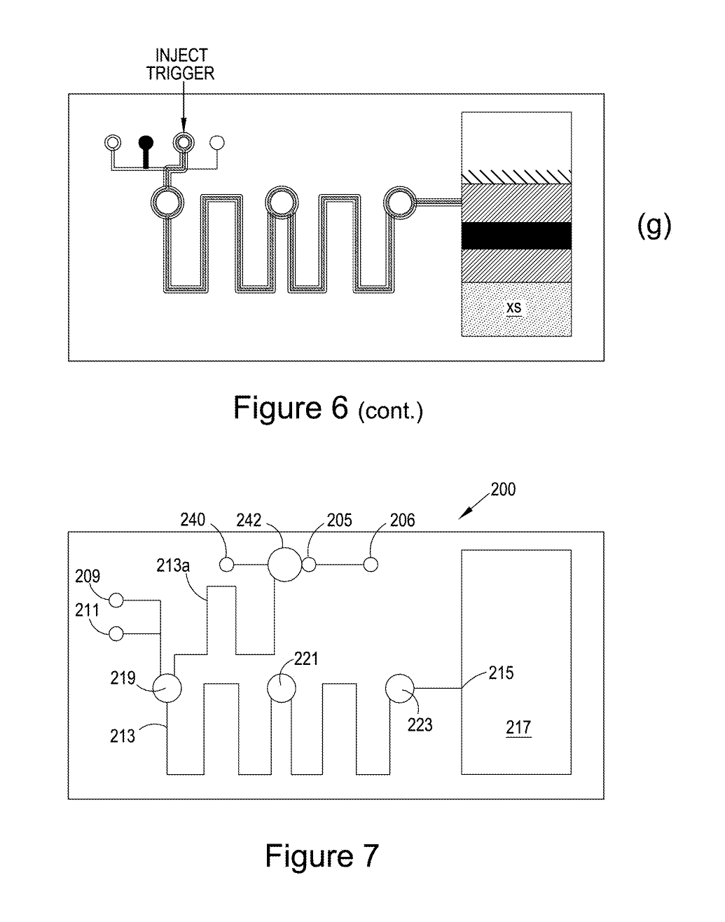

A sample of blood from a patient (optionally mixed with an anti-coagulant and/or diluted, so as to facilitate passage of the sample along the fluid conduit without coagulation occurring) is first injected through the sample inlet 5 using a conventional syringe. The sample is flushed through the fluid conduit 13, filling each of the chambers 19, 21 and 23. Any excess sample (XS) passes through the waste outlet 15 and to the waste sink 15 (see FIGS. 6(a)-(b)).

The cassette 1 is then placed into the slot in the reader device (as shown in FIG. 4) and the needles 29 extended (by way of electronic motors, not shown) to pierce the silicone layer 27 and open the respective inlets 5, 7, 9 and 11. Once the cassette has been coupled to the reader device in this way, the remaining steps of the multiplexed assay are automated. Accordingly, from the user's perspective, the assay requires a only a single step of injecting the sample, and the assay is, in effect, “pseudo-homogeneous”. A user of the system 100 therefore requires no special training in order to carry out the multiplexed assay.

The progress of the assay is shown schematically in FIG. 6.

Immediately following insertion of the cassette 1 in to the slot of the reader device 130, the cassette is in the configuration shown in FIG. 6(b), with the entire fluid conduit (including the chambers) filled with the sample. As mentioned above, each of the chambers 19, 21 and 23 are filled with the analyte-specific antibody probes bound to polystyrene beads, specific to each of three thyroid hormones. A proportion of any of each of the hormones are immobilized in the chambers by the analyte specific probes, in an amount proportional to their concentrations within the sample.

The assay system 100 may wait for a minimum period of time to ensure that immobilization is complete.

In a next step, pump 110 activates, to pump a washing fluid (buffer solution) through the inlet 5. This washes any non-immobilized sample through the fluid conduit 13 and into the waste sink 17 (FIG. 6(c)).

In a next step, pump 112 activates, to pump a labelling fluid through the reagent inlet 7 and along the fluid conduit (FIG. 6(d)). The labelling fluid displaces the washing fluid in each of the chambers. The labelling fluid comprises a solution of each of three analyte-specific antibody probes, which are antibodies specific to each of the three thyroid hormones, labelled with acridinium ester moieties (1). The labelled antibodies bind to and are immobilized by any of the corresponding analyte already immobilized in each of the sample chambers. Thus, each chamber contains immobilized analyte-specific antibodies, a proportion of which have a respective thyroid hormone bound thereto, with labelled analyte-specific probes bound to the immobilized analyte.

The fluid conduit is again washed (by actuation of pump 110) to flush excess unbound labelled antibodies out of the chambers and into the waste sink (FIG. 6(e)).

In a next step, with the aperture 122 open adjacent to the chamber 19, an aliquot of triggering fluid (alkaline hydrogen peroxide solution) is pumped through inlet 9, by pump 114. The triggering fluid may optionally be provided pre-mixed, or hydrogen peroxide may be mixed with an alkali (e.g. NaOH) so as to form the triggering fluid immediately prior to, or during the process of introducing the triggering fluid into the fluid pathway. An amount is pumped along the fluid conduit sufficient to fill the chamber 19 furthest upstream in the fluid conduit 13, and to thereby trigger the chemiluminescent C9 cleavage reaction described above. An optical measurement may thus be obtained from the chamber 19, the intensity of chemiluminescent light detected being proportional to the amount of immobilized analyte present in the chamber.

The pumping of the triggering fluid is discontinued, consequent to a step change in the measured conductivity from the electrodes 37, before the leading end of the aliquot reaches the second chamber 21.

In a next step, with the aperture 122 now open adjacent to the second chamber 21, further triggering fluid is pumped into the fluid conduit through the inlet 9 (FIG. 6(f)). It is to be understood that equally, the initial aliquot of triggering fluid could be advanced along the fluid conduit by pumping washing fluid through the inlet 5.

The triggering fluid fills the second chamber 21 and triggers the chemiluminescent reaction therein, and an optical measurement is taken from the second chamber. The pumping of the triggering fluid is discontinued, consequent to a step change in the measured conductivity from the electrodes 38, before the leading end of the aliquot reaches the third chamber 23.

Finally, the triggering fluid is pumped through the fluid conduit so as to fill and trigger chemiluminescence in the third chamber (FIG. 6(g)).

After each optical measurement raw measured data is output from the PMT 118 to the computing device 132. The computing device then processes the data and outputs processed quantitative measurement data, in the form of analyte concentration, to the display (in some embodiments, data is output to the display after all of the measurements are complete and processed).

The multiplexed assay is then complete and all of the reagents used are contained either within the fluid conduit or the waste sink. The cassette may thus be removed from the reader device and disposed of.

In the example shown, the further fluid inlet 11 is not required. However, in alternative embodiments, it may be desirable to use a different type of chemical label attached to an analyte-specific probe which is specific to an analyte immobilized in the second and/or the third chamber. Thus, an aliquot of a further triggering fluid, formulated to trigger a different type of chemical label, may be pumped into the fluid conduit via the inlet 11, so as to trigger a chemiluminescent reaction in the second and/or third chamber. This methodology is made possible because the convoluted regions of the fluid conduit between the chambers have a greater volume than the volume enclosed by the chambers themselves.

In other embodiments (not shown) one or more reagent inlets may communicate directly with each chamber. This enables more than one chemiluminescent reaction to be triggered and this more than one measurement to be acquired from each chamber.

The cassette 1 may also be used for a competitive assay. In that case, the sample may be mixed with a conjugate fluid prior to injection into the fluid conduit 13. The conjugate fluid may comprise known amounts of each of three analyte-chemical label conjugates, corresponding to the immobilized analyte-specific probes present in each of the chambers 19, 21 and 23. Once the sample/conjugate mixture has been passed along the fluid conduit, the chambers will each contain immobilized analyte from the sample and labelled immobilized analyte from the conjugate fluid, in amounts proportional to their relative concentrations in the mixed fluid. Consequently, when a triggering fluid is pumped through the fluid conduit (as described above with reference to FIGS. 6(e)-(g)), the intensity of the chemiluminescent signal is inversely proportional to the relative concentrations of the analyte from the sample and from the conjugate fluid.

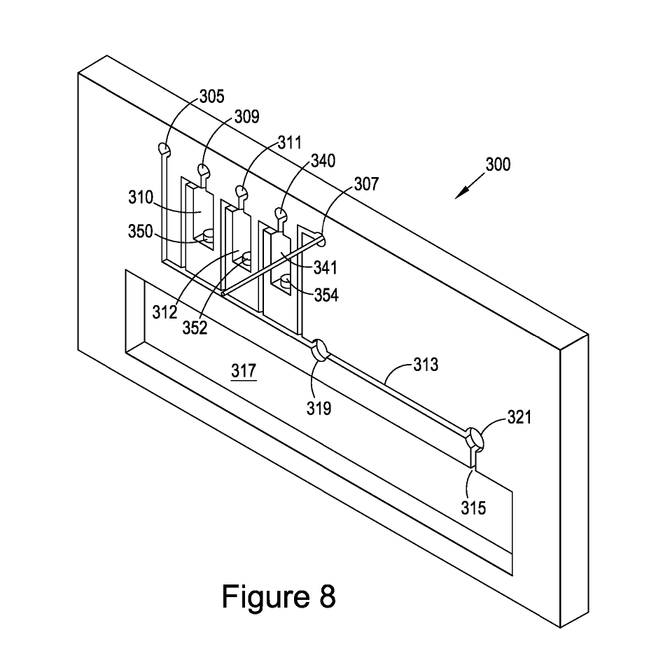

FIG. 7 shows a schematic diagram of an assay cassette 200 specifically adapted to perform a competitive assay. Features in common with cassette 1 are provided with like reference numerals, incremented by 200. The layout and relative positions of the inlets are not to scale.

The cassette 200 has a sample inlet 205 to a mixing chamber 242. A washing fluid inlet 206 is positioned upstream of the sample inlet, and a conjugate fluid inlet 240 communicates with the fluid conduit at the mixing chamber.

The reagent inlets 209 and 211 are positioned in the same positions with respect to the cassette 200 as inlets 9 and 11 with respect to cassette 1. Similarly, the washing fluid inlet 206 and the conjugate fluid inlet 240 are in the same positions as reagent inlets 5 and 7 of the cassette 1. This arrangement enables the reader device 130 to be used with the both the cassette 1 and the cassette 200.

The reagent reservoir 102 is again filled with the washing solution, and the reservoirs 106 are filled with a triggering solution or solutions. For use with the cassette 200, the reagent reservoir 104 is filled with a conjugate fluid.

In use, an operative injects a sample sufficient to fill the mixing chamber. The mixing chamber thereby functions to control/limit the amount of sample injected and thus aids in the quantification of the assay. The cassette 200 is then inserted into the reader device. A known amount of the conjugate fluid is then injected through the conjugate inlet 240, from the reservoir 104. The sample and conjugate fluids mix in the mixing chamber and as they flow along the portion 213a of the fluid conduit, and the competitive assay performed in the manner described above. The cassette may optionally comprise fewer chambers (for example one or two chambers) or a greater number of chambers.

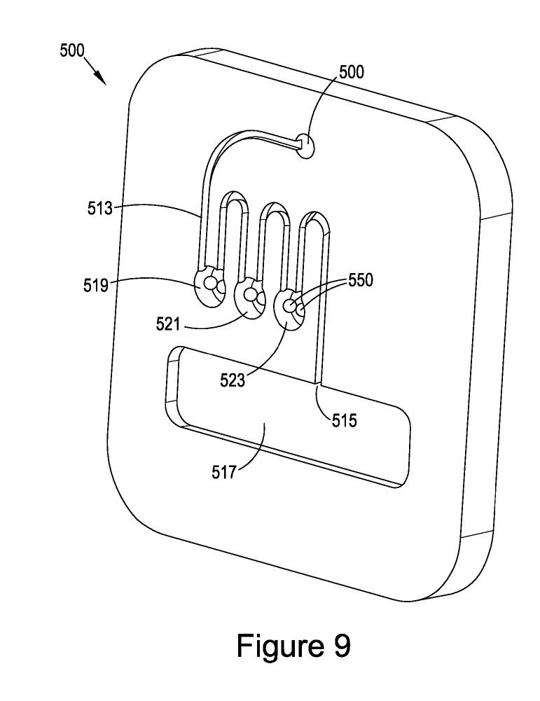

FIG. 8 shows a still further embodiment of an assay cassette 300 in accordance with the invention. Features in common with cassette 1 are provided with like reference numerals, incremented by 300.

The assay cassette includes a sample inlet 305 leading to a waste outlet 315 via a fluid conduit 313. The fluid conduit includes two chambers 319, 321 in series. The cassette 300 also has a washing fluid inlet 307 and reagent inlets 309, 311 and 340. Each of the reagent inlets leads to a corresponding reagent reservoir 310, 312, 341. Each mixing chamber contains a soluble tablet 350, 352, 354. Tablets 350 and 352 include a labelling agent (comprising labelled analyte-specific probes for the analytes to be immobilized in each of the chambers) and a triggering agent, respectively. Tablet 354 includes a conjugate agent. In use and as required by the assay in question, washing fluid is injected into the reagent inlets, which dissolves the tablet in the adjacent reservoir to form a respective reagent solution in a reservoir in the cassette. This enables the assay to be conducted using only a single type of reagent (washing fluid) stored within the corresponding reader device.

The cassette is also configured to enable either a sandwich or a competitive assay to be performed. Consequently, a sandwich assay can be performed using the reagent inlets 309 and 311, so that labelling fluid and triggering fluid are formed. In that case, the inlet 340, reservoir 341 and tablet 354 are not required. Alternatively, a competitive assay may be performed using the reagent inlets 340 and 311, so that a conjugate fluid and a triggering fluid are formed. In that case, inlet 309, reservoir 310 and tablet 350 are not required.

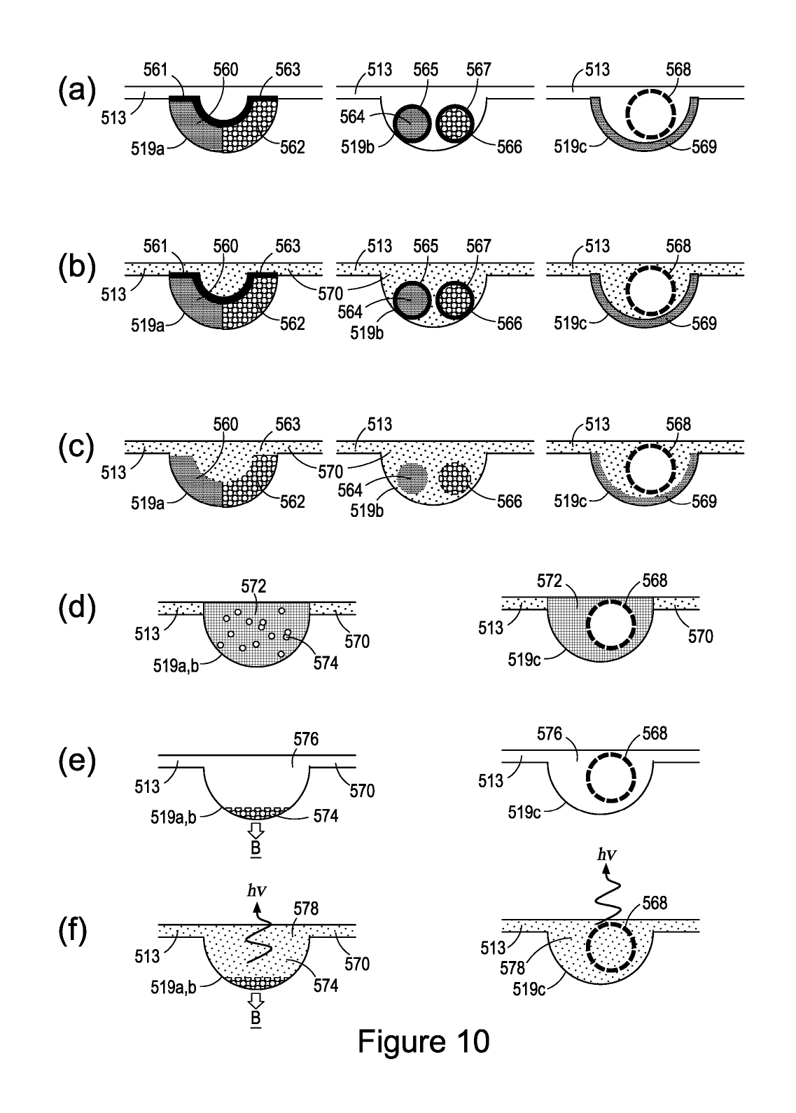

FIG. 9 shows a still further embodiment of an assay cassette 500 in accordance with the invention. Features in common with cassette 1 are provided with like reference numerals, incremented by 500.

The cassette 500 has fluid conduit 513 extending from a sample inlet 505 to a waste outlet 515 to a waste sink 517. Three chambers 519, 521 and 523 are positioned in series in the fluid conduit. Each chamber includes assay specific reagents 550 in solid form. In the example shown, the cassette 500 is configured to conduct a multiplexed competitive assay and each chamber contains two beads; one comprising conjugate and the other comprising immobilized analyte-specific probes on magnetic beads. In alternative embodiments, a chamber may contain a single combined bead or tablet, or a tablet (e.g. coating or adhered to a chamber wall) for one type of reagent and bead or tablet of another type. It will also be understood that the cassette may be configured to perform a sandwich assay, in which case a conjugate tablet/bead would not be present.

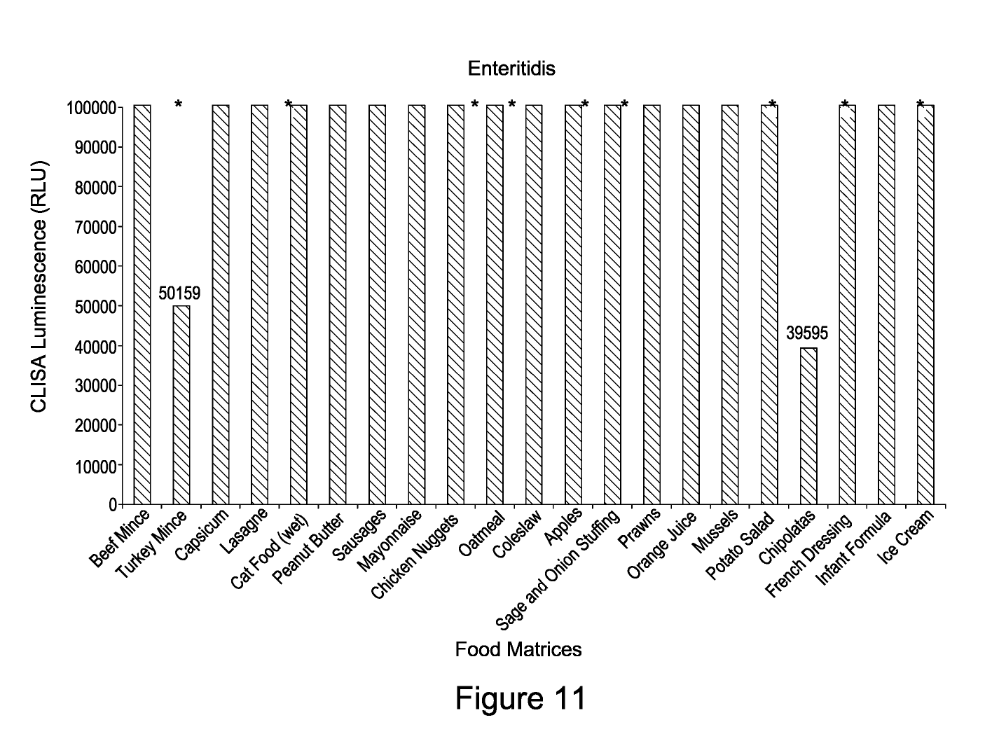

FIG. 10 schematically shows the steps of a competitive assay within a chamber of an assay cassette such as cassette 500.

In order to operate a competitive assay, precise amounts of sample must react with precise amounts of antibody-conjugated target. This is a non-trivial requirement and the accuracy of a competitive assay may be compromised by variations for example in the sample volume or the volume of sample or other reagents which are delivered to a chamber.

Such problems may be addressed by providing reagents in solid form, as a tablet placed in the well or coating a wall of the chamber, or as a bead placed in the chamber. Three such alternatives are shown in FIG. 10a. A wall of chamber 519a is coated with a tablet 560 comprising conjugate and a tablet 562 comprising assay-specific probe immobilized on magnetic microbeads.

The tablet 560 comprises a conjugate-matrix composition, in which the conjugate is mixed with a soluble matrix formed from trehalose.

The tablet 562 comprises immobilized analyte-specific probe, immobilized on magnetic microbeads, in a trehalose soluble matrix.

Each of the tablets 560, 562 is provided with a soluble coating 561, 563 of Eudraglit L 12.5 (which is an anionic co-polymer material based on methacrylic acid and methyl methacrylate). Eudraglit is a trade mark of Evonic Industries AG. The coating and the wall of the chamber 519a to which the tables are bound together protect the inner soluble matrix. The trehalose matrix and the coating together protect against long term degradation of the respective reagents.

In addition, and as described further below, the coatings 561, 563 retard solublisation of the trehalose matrix tablet for approximately one minute, to allow the respective liquid sample to fill the chamber, and before the reagents are released. When the reagents are released, fluid is not flowing through the fluid conduit 513 or the chamber 519a-c and so the chamber will contain precise and desired quantities of, for example, conjugate and sample.

An alternative arrangement is shown in chamber 519b, which contains the reagents in the form of a conjugate-containing bead 564 and an analyte-specific probe-containing bead 566. Again, each bead 564, 566 is provided with a Eudraglit L 12.5 soluble coating 565, 567.

Another alternative arrangement is shown in chamber 519c, which contains a single polystyrene bead 568, coated with analyte-specific probe (e.g. antibody). A coated tablet 569 comprises the conjugate in a trehalose matrix.

The beads 564, 566 and 568 are larger than the diameter of the conduit 513 and thereby retained in their chamber. The tablets 560, 562, 569 are adhered to the wall of the chamber.

FIG. 10b shows a next step of a competitive assay to be conducted in a chamber 519a-c. Sample fluid 570 is injected through the sample inlet, along the conduit 513 and into the chamber. Where a multiplexed assay is to be conducted, all chambers would be filled and excess sample would flow out of the outlet 515 and into the waste sink 517. At this stage, the inner matrix part of the respective tablets or beads remain protected by the soluble coating.

In a next step, shown in FIG. 10c, once the flow of sample fluid 570 has ceased, the soluble coatings 561, 563, 565, 567, 569 are eroded by the assay solvent (e.g. water) and, subsequently and typically more rapidly, the trehalose matrix is dissolved (FIG. 10d). This releases both the conjugate (thus forming a conjugate fluid 572) and, from the tablet 562 or the bead 566, the magnetic microbead immobilized analyte specific probe 574. Optionally, dissolution and mixing may be facilitated by mechanically agitating and/or heating the fluid in the chamber (apparatus omitted for clarity).

Immobilization of any analyte within the sample, and corresponding labelled analyte within the conjugate fluid, may take place during an incubation period of ca. 20 minutes.

A wash fluid 576 may then be flushed through the conduit 513 so as to wash any unbound sample from the chamber (FIG. 10e). During this process, a magnetic field B may be applied to the chambers 519a,b comprising the magnetic microbeads, so as to retain them therein. The immobilized materials are retained within the chamber 519c, by virtue of the size of the bead 568. Again, mechanical agitation may be applied during this procedure.

Optionally, flow of washing fluid may be paused and the magnetic field may be released to allow the microbeads to more thoroughly mix with the washing fluid, before the field B is reapplied and the flow recommenced. This may be conducted several times, optionally whilst mechanical agitation is applied.

It will be understood that when the magnetic microbeads are free to mix with fluids in the chamber, since fluid is not flowing along the fluid conduit, beads or conjugate fluid may only leave the chamber by diffusion into the conduit. By selection of the conduit diameter and the chamber volume, it can be ensured loss of reagent by diffusion in this way has negligible effect on the assay.

In a final step, triggering fluid is flushed along the conduit so as to fill the chambers. Where a multiplexed assay is to be conducted, an amount of triggering fluid (comprising a peroxide solution) sufficient to flow into and fill a single chamber in turn, is caused to flow along the conduit.

As shown in FIG. 10f, once the chambers are filled with triggering fluid 578, it reacts with acridinium ester labels provided on labelled analyte bound to the beads 574, 568 (which originate from the conjugate) and a quantitative signal hv may be acquired.

### Food Tests for Salmonella.

An assay cassette in accordance with the invention has also been used to obtain measurements of a range of Salmonella species in a wide variety of standard food types under normal food testing conditions.

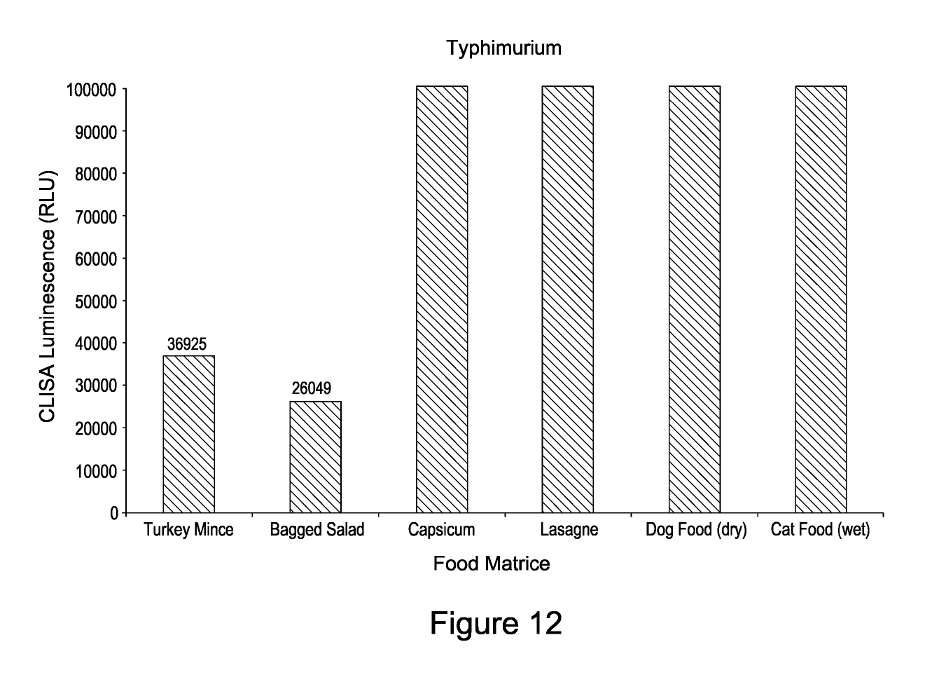

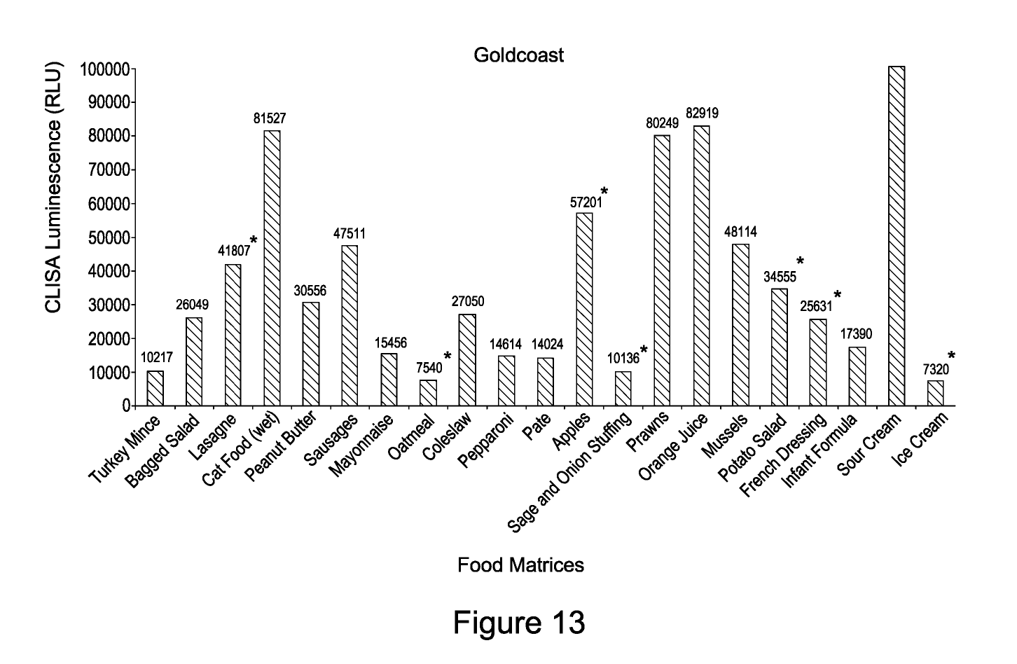

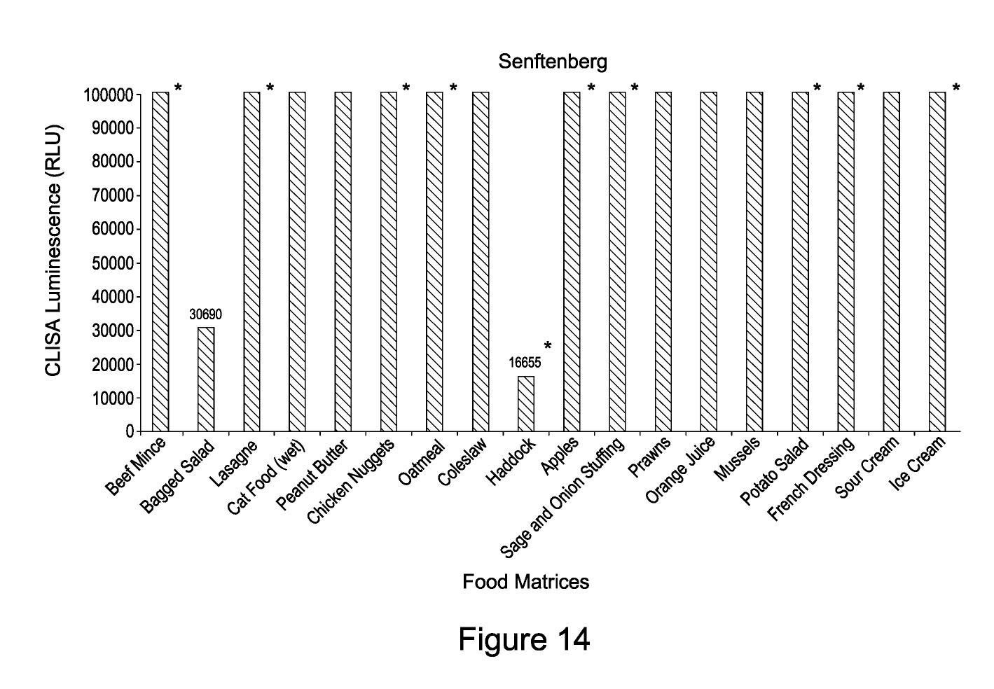

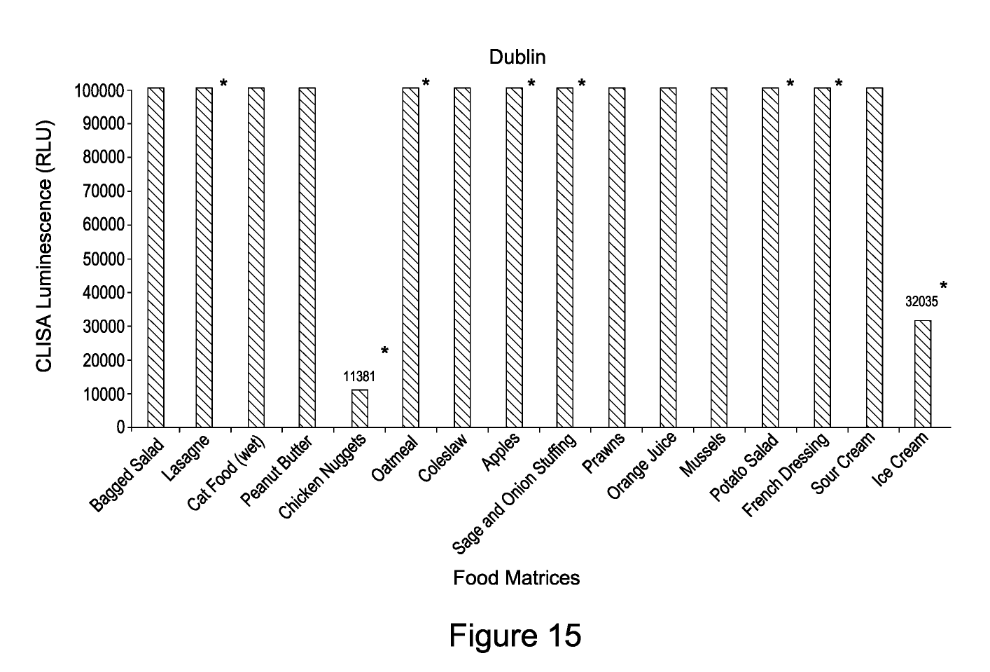

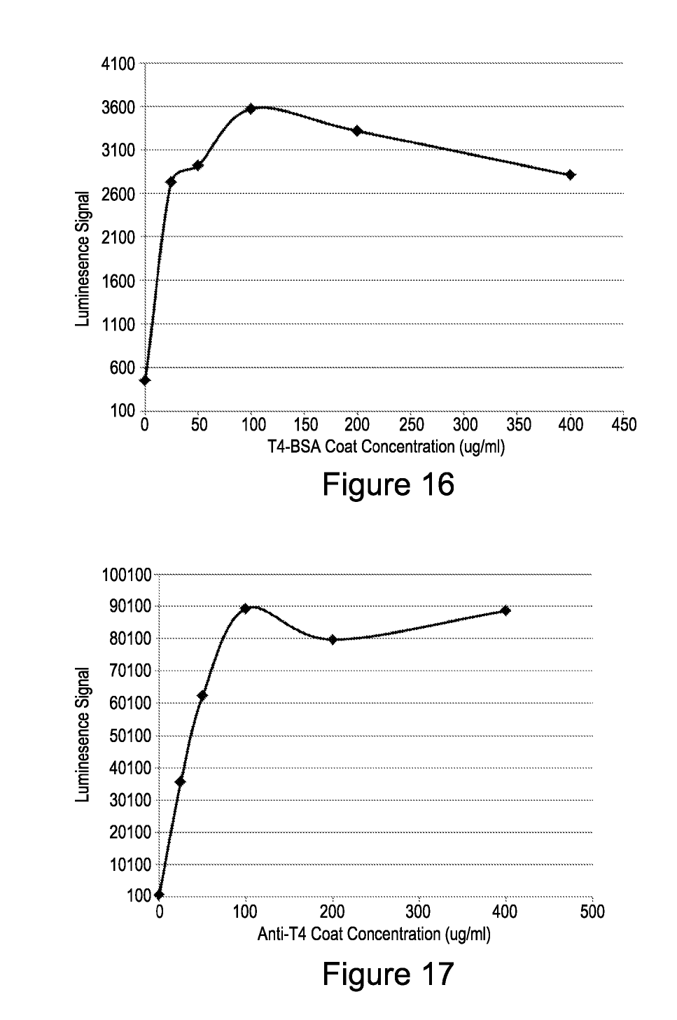

Results showing concentrations of Salmonella enteritidis, typhimurium, goldcoast, senftenburg and dublin in a range of food samples are shown in FIGS. 11-15, respectively.

Data were obtained using a polyclonal antiserum from KPL Inc, USA, in a sandwich immunoassay format. The antiserum was labelled with 2′,6′-dimethylcarbonylphenyl-10-sulfopropylacridinium-9-carboxylate 4′-NHS ester (DMAE—Toronto Research Chemicals Inc, Canada).

Test data values recorded are in Relative Light Units (RLU) which are at least 2× the standard deviation of the negative control value, i.e. the mean negative control+2×SD=0 RLU, therefore, any result above the y-axis is a statistically significant result.

An asterisk (*) over a food type in the Figures indicates that the bacterium used was at a low number and was stressed (44 foods out of 109 tested).

The tested Salmonella bacteria cover approximately 75% of the Salmonellas found in foods, including a difficult spore forming type (dublin) that is the subject of significant interest at present. The test values for any bacterium varies across food types because each food matrix impacts the growth rate of each bacterial serotype, however, the assay cassette has been shown to be capable of detecting the present, or absence, of each species of Salmonella in a 25 gram food sample.

Various modifications may be made to embodiments herein described without departing from the scope and spirit of the invention. For example, the reader device may be configured to acquire a different type of optical measurement, or the system may be configured to allow for measurement of an electrical property of material within the chambers. The cassettes may be provided with a different number of chambers, and/or a different arrangement of mixing chambers, reagent reservoirs, reagent inlets and so forth. Control over the reagents may be provided by a different type of pump, or fluid may be conveyed along the fluid conduit at least in part by capillary action. The system may be provided with other means of automation, such as injection needles adapted to be moved, so as to be capable of injecting a fluid through more than one inlet.

### THYROXINE (T4) Competitive Assay

### Bead Coating and Assay Procedure.

The methodology described above in relation to FIG. 10 may be applied to conduct a thyroxine competitive assay, as follows.

### Materials and Equipment

Paramagnetic particles (magnetic microbeads) 1-2 micron diameter—Polysciences Cat No 18879, Lot No 501507
Stock concentration=25 mg/ml
T4-BSA—US Biological Cat No. T5460-20, Lot No L14091015
   Stock concentration=0.9 mg/ml=13.5 nmol/ml
T4-HRP—US Biological Cat No. T5460-30, Lot No L14091014
   Stock Concentration=0.4 mg/ml=8.9 nmol/ml
Anti-T4 Antibody—AbD Serotec Cat No 8959-9831, Batch No 0709
   Stock Concentration=1 mg/mi
Acridinium ester labelling kit—Cayman Chemical Cat No 200201
Supersignal Pico HRP Luminesce substrate—Pierce Cat No 37070

### BMG PolarStar Luminescence Plate Reader

### Bead Coating Procedure

Magnetic beads were coated by diluting stock beads to a concentration of 1 mg/ml in 100 mM carbonate, pH 9.6 buffer along with an appropriate concentration of coating protein (either T4-BSA or Anti-T4 antibody) and incubating overnight at 4° C. with gentle agitation.

For example for a 100 μg/ml protein coating: 40 μl bead stock+111 μl T4-BSA stock+849 μl 100 mM carbonate, at pH9.6 in Buffer.

In a further example, a protein coating was provided using: 40 μl bead stock+100 μl anti-T4 stock+860 μl 100 mM carbonate, at pH9.6 in Buffer.

For saturation experiments beads were coated with 0, 25, 50, 100, 200 and 400 μg/ml of T4-BSA or anti-T4 and detected with 40 pmol/ml anti-T4-Acr ester or T4-HRP respectively.

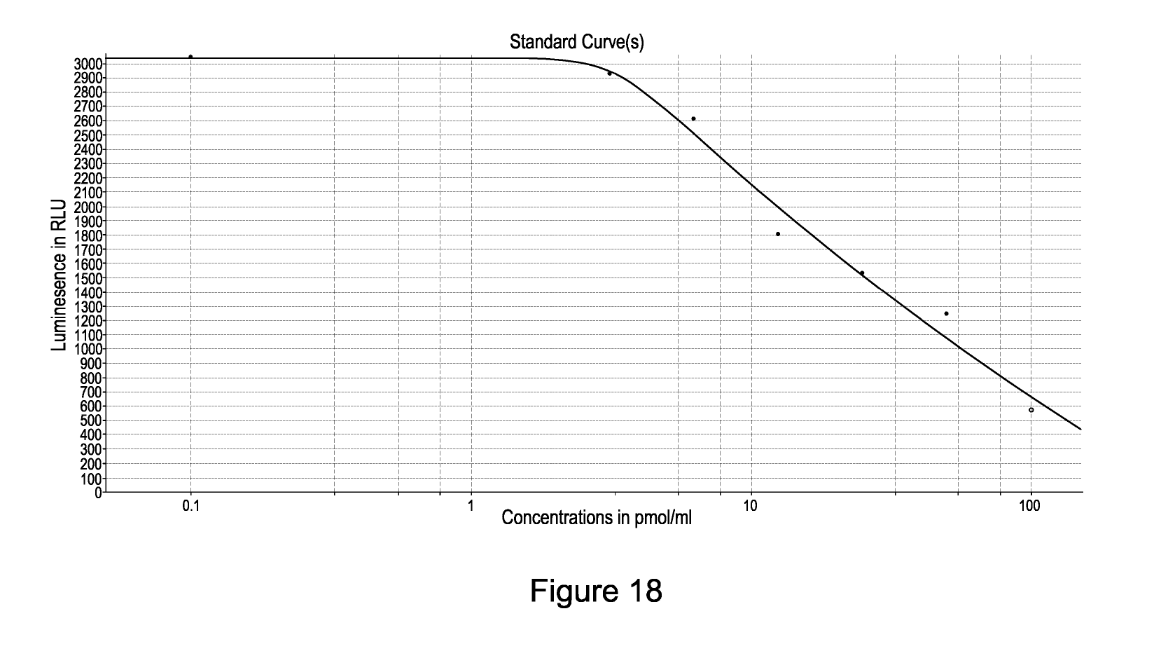

The saturating protein concentration was shown to be 100 μg/ml with both T4-BSA coating (FIG. 16) and Anti-T4 coating (FIG. 17)

### T4 Competitive Assay Procedure

Magnetic beads coated at the saturating concentration of either T4-BSA or Anti-T4 were incubated with 40 pmol/ml anti-T4-Acr ester or T4-HRP respectively and increasing concentrations of T4-BSA.

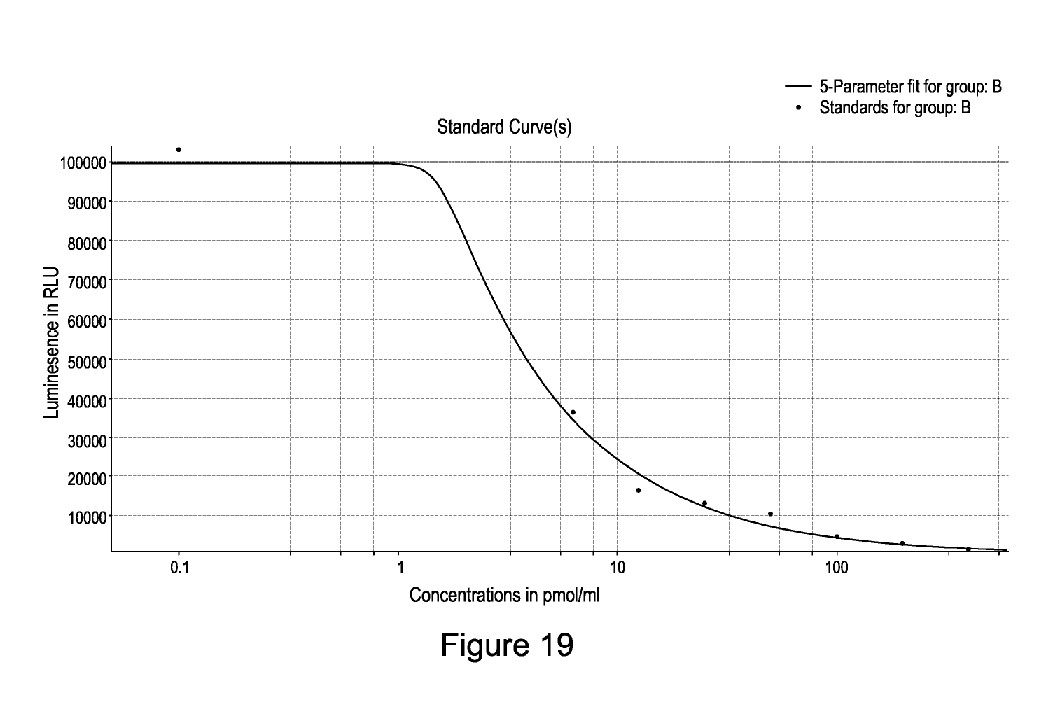

T4-BSA coated beads showed competitive inhibition of signal over the range of approximately 1 to 100 pmol/ml T4-BSA (FIG. 18) and anti-T4 coated beads showed inhibition over the range of approximately 1 to 400 pmol/ml (FIG. 19).

1. An assay cassette for use in performing a heterogeneous assay, the assay cassette comprising
a fluid conduit extending between a sample inlet and a waste outlet; and
at least two chambers disposed in series along the fluid conduit, downstream of the sample inlet and upstream of the waste outlet;
each chamber comprising an immobilized analyte-specific probe, and adapted to enable acquisition of a respective measurement relating to the amount of immobilized analyte in each chamber, in use.

2. An assay cassette according to claim 1, comprising one or more reagent inlets in fluid communication with the fluid conduit, through which reagent may be introduced into the fluid conduit.

3. An assay cassette according to claim 1, wherein one or more said reagent inlets are in fluid communication at or immediately upstream of a said chamber.

4. An assay cassette according to claim 1, wherein the analyte-specific probe in each chamber is immobilized by binding, chemically or physically, to a wall or walls of the chamber.

5. An assay cassette according to claim 1, wherein the analyte-specific probe in each chamber is immobilized by binding to a solid support medium contained in the chamber.

6. An assay cassette according to claim 5, wherein the solid support medium comprises an absorbent or adsorbent material, such as a porous or molecular sieve material;
a particulate material, such as beads or a single bead; or
magnetic particles, such as magnetic microbeads.

7. An assay cassette according to claim 5, wherein the analyte-specific probe is immobilized by incorporation into a matrix formed by a solid support medium.

8. An assay cassette according to claim 5, wherein the solid support medium is retained in each chamber by virtue of
the particle size of the solid support medium in comparison to the dimensions of the outlet/inlet of the chamber; or
in use, by one or more magnets.

9. An assay cassette according to claim 1, comprising one or more reagents, in solid or liquid form, pre-packed on the cassette during manufacture.

10. An assay cassette according to claim 9, comprising one or more assay specific reagents, selected from one or more labelling fluids or one or more conjugate fluids, in addition to said immobilized analyte-specific probe(s).

11. An assay cassette according to claim 9, wherein the one or more reagents, are provided within a soluble matrix, which is capable of dissolving in the assay solvent so as to release the reagent(s) into solution in use.

12. An assay cassette according to claim 11, wherein one or more said chambers contain a tablet or bead comprising a reagent-matrix composition, the reagent-matrix composition comprising one or more reagents in the soluble matrix.

13. An assay cassette according to claim 12, wherein the/each tablet or bead, is provided with a soluble coating, capable of being eroded by a reagent or a sample fluid used in the assay.

14. An assay cassette for use in performing an assay, comprising
a fluid conduit extending between a sample inlet and a waste outlet; and
a chamber disposed along the fluid conduit, downstream of the sample inlet and upstream of the waste outlet;
the chamber comprising a tablet or bead, the tablet or bead comprising a reagent-matrix composition;
the reagent-matrix composition comprising a reagent (or reagents), in a soluble matrix, which is capable of dissolving in a reagent or a sample fluid used in the assay, so as to release the reagent(s) into solution in use;
the tablet or bead provided with a soluble coating, capable of being eroded by a reagent or a sample fluid used in the assay, so as to expose the reagent-matrix composition, in use.

15. An assay cassette according to claim 14, wherein the tablet or bead comprises a soluble matrix and soluble coating which are water soluble.

16. An assay cassette according to claim 14, wherein the tablet or bead comprises a soluble matrix which is a di-, tri- or oligosaccharide, such as trehalose.

17. An assay cassette according to claim 14, wherein the soluble coating of the tablet or bead is a water-soluble polymer, such as poly(methacrylic acid-co-methyl methacrylate).

18. An assay cassette according to claim 14, for use in performing a competitive assay, wherein the tablet or bead comprises a conjugate-matrix composition, the conjugate-matrix composition comprising a chemical label bound to a control analyte, in a soluble matrix.

19. An assay cassette according to claim 14, wherein the tablet or bead comprises an immobilized analyte-specific probe, immobilized on a particulate solid support medium, in the soluble matrix.

20. An assay cassette according to claim 19, wherein the particulate solid support medium comprises magnetic particles to which the analyte-specific probe is immobilized.

21. An assay cassette according to claim 1, comprising a labelling fluid, comprising a chemical label may be bound to an analyte probe, the labelling fluid capable of labelling each of the analytes immobilized in the respective chambers in the cassette, in use of the cassette.

22. An assay cassette according to claim 21, wherein the chemical label comprises an acridinium or acridan ester moiety.

23. An assay cassette according to claim 1, wherein the enclosed volume of the portion or portions of the fluid conduit between adjacent chambers is greater than the defined volume of the chamber immediately upstream thereof.

24. An assay cassette according to claim 23, comprising one or more convoluted fluid conduit portions between chambers, so as to provide sufficient enclosed volume on a compact assay cassette.

25. An assay cassette according to claim 1, wherein a face of each said chamber is sealed with an optically transparent layer of material.

26. An assay cassette according to claim 1, configured to detect one of more types of analyte selected from; proteins, peptides, antibodies, nucleic acid, microorganisms (such as bacteria and viruses), chemical agents, toxins, pharmaceuticals, metabolites, cellular moieties.

27. An assay cassette according to claim 1, comprising a mixing portion of the fluid conduit upstream of the chambers, adapted to facilitate mixing of a conjugate fluid and a sample fluid, in use.

28. An assay cassette according to claim 27, wherein the mixing portion comprises a mixing chamber, disposed in the fluid conduit between the sample inlet and the most upstream chamber.

29. An assay cassette according to claim 1, wherein the fluid conduit is dimensioned such that fluid must be pumped in order to flow along the fluid conduit.

30. A method of performing an assay, comprising
introducing a sample fluid into a fluid conduit through a sample inlet to the fluid conduit;
causing the sample fluid to pass along the fluid conduit, and into each of at least two chambers disposed in series along the fluid conduit;
using immobilized analyte-specific probes present in each of the chambers to immobilize at least a portion of any of the specific analyte or analytes which are present in the sample fluid within the respective chambers; and
acquiring a measurement of each of the chambers, relating to the amount of immobilized analyte therein.

31. A method according to claim 30, comprising performing the assay at the point of care, or at the point of use, as the case may be.

32. A method according to claim 30, comprising one or more steps of each of:
washing, so as to wash excess or non-immobilized material out of a chamber;
triggering, so as to trigger a measurable response from material, such as a chemical label, present in a chamber.

33. A method according to claim 30, comprising mechanically agitating the cassette or a chamber thereof, during one or more steps of the assay.

34. A method according to claim 30, comprising triggering a measureable response from material present in a first chamber, acquiring a measurement from the first chamber, and subsequently triggering a measurable response from a second chamber and acquiring a measurement from the second chamber.

35. A method according to claim 34, wherein the measureable responses are triggered in the first and second chambers using a triggering fluid, optionally a single aliquot of the triggering fluid.

36. A method according to claim 30, comprising pumping one or more fluids along, and/or into, the fluid conduit.

37. A method according to claim 30, comprising introducing a sample fluid into the fluid conduit, followed by an aliquot of a labelling fluid, followed by an aliquot of a triggering fluid, the method optionally comprising introducing into the fluid conduit an aliquot of washing fluid after the sample fluid and/or labelling fluid and/or triggering fluid.
